(19) United States
(12) Patent Application Publication
(10) Pub. No.: US 2026/0248562 A1
(43) Pub. Date: Aug. 27, 2026

(54) METHODS AND SYSTEMS FOR DISPLAYING PREOPERATIVE AND INTRAOPERATIVE IMAGE DATA OF A SCENE

(71) Applicant: Proprio, Inc., Seattle, WA (US)

(72) Inventors: Eve Maria Powell, Bellevue, WA (US); David Lee Fiorella, Seattle, WA (US); Camille Cheli Farley, Kirkland, WA (US); David Franzi, Seattle, WA (US); Nicholas Matthew Miclette, Seattle, WA (US); Adam Gabriel Jones, Seattle, WA (US)

(21) Appl. No.: 19/390,406

(22) Filed: Nov. 14, 2025

Related U.S. Application Data
(63) Continuation of application No. 17/864,065, filed on Jul. 13, 2022, now abandoned.
(60) Provisional application No. 63/221,428, filed on Jul. 13, 2021.

(51) Int. Cl. A61B 34/20; A61B 34/00

(52) U.S. Cl. CPC A61B 34/20; A61B 34/25; A61B 90/06; A61B 90/361; A61B 90/37; A61B 2034/2065; A61B 2090/062; A61B 2090/367; A61B 2090/3762; A61B 2562/046

(57) ABSTRACT

Mediated-reality imaging systems, methods, and devices are disclosed herein. In some embodiments, an imaging system includes a camera array configured to (i) capture intraoperative image data of a surgical scene in substantially real-time and (ii) track a tool through the scene. The imaging system is further configured to receive and/or store preoperative image data, such as medical scan data corresponding to a portion of a patient in the scene. The imaging device can register the preoperative image data to the intraoperative image data, and display the preoperative image data and a representation of the tool on a user interface, such as a head-mounted display.

### CROSS-REFERENCE TO RELATED APPLICATIONS

This application is a continuation of U.S. patent application Ser. No. 17/864,065, filed Jul. 13, 2022, and titled “METHODS AND SYSTEMS FOR DISPLAYING PREOPERATIVE AND INTRAOPERATIVE IMAGE DATA OF A SCENE,” which claims the benefit of priority to U.S. Provisional Patent Application No. 63/221,428 filed Jul. 13, 2021, and titled “METHODS AND SYSTEMS FOR DISPLAYING PREOPERATIVE AND INTRAOPERATIVE IMAGE DATA OF A SCENE,” the disclosure of each of which is incorporated herein by reference in its entirety.

### TECHNICAL FIELD

The present technology generally relates to methods and systems for displaying previously-captured image data, such as preoperative medical images (e.g., computed tomography (CT) scan data).

### BACKGROUND

In a mediated reality system, an image processing system adds, subtracts, and/or modifies visual information representing an environment. For surgical applications, a mediated reality system may enable a surgeon to view a surgical site from a desired perspective together with contextual information that assists the surgeon in more efficiently and precisely performing surgical tasks. When performing surgeries, surgeons often rely on preoperative three-dimensional images of the patient's anatomy, such as computed tomography (CT) scan images. However, the usefulness of such preoperative images is limited because the images cannot be easily integrated into the operative procedure. For example, because the images are captured in a preoperative session, the relative anatomical positions captured in the preoperative images may vary from their actual positions during the operative procedure. Furthermore, to make use of the preoperative images during the surgery, the surgeon must divide their attention between the surgical field and a display of the preoperative images. Navigating between different layers of the preoperative images may also require significant attention that takes away from the surgeon's focus on the operation.

The present technology generally relates to methods and systems for generating a real-time or near-real-time three-dimensional (3D) virtual perspective of a scene for a mediated-reality viewer, and registering previously-captured image data, such as preoperative medical images (e.g., computed tomography (CT) scan data), to the 3D virtual perspective.

### BRIEF DESCRIPTION OF THE DRAWINGS

Many aspects of the present disclosure can be better understood with reference to the following drawings. The components in the drawings are not necessarily to scale. Instead, emphasis is placed on clearly illustrating the principles of the present disclosure.

FIG. 1 is a schematic view of an imaging system in accordance with embodiments of the present technology.

FIG. 2A is a perspective view of a surgical environment employing the imaging system of FIG. 1 for a surgical application in accordance with embodiments of the present technology.

FIG. 2B is an isometric view of a portion of the imaging system of FIG. 1 illustrating four cameras of the imaging system in accordance with embodiments of the present technology.

FIG. 3 illustrates a user interface visible to a user of the system of FIG. 1 in accordance with embodiments of the present technology.

FIGS. 4A-4C illustrate the user interface of FIG. 3 including the display of a ruler in accordance with embodiments of the present technology.

FIGS. 5A and 5B illustrate the ruler of FIGS. 4A-4C in accordance with additional embodiments of the present technology.

FIGS. 5C and 5D illustrate different views of the user interface of FIGS. 4A-4C in accordance with embodiments of the present technology.

FIGS. 5E and 5F are views of the ruler of FIGS. 4A-4C in accordance with additional embodiments of the present technology.

FIGS. 6A-6D illustrate an instrument approaching, entering, and moving through 3D image data in accordance with embodiments of the present technology.

FIGS. 7A-7D illustrate 3D image data including different slicing planes in in accordance with embodiments of the present technology.

FIG. 8 is an enlarged illustration of 3D image data of a vertebra shown as translucent in accordance with embodiments of the present technology.

FIGS. 9A-9C illustrate a user interface visible to a user of the system of FIG. 1 in accordance with additional embodiments of the present technology.

FIGS. 10A-10D illustrate 3D image data and an instrument approaching, entering, and moving through the 3D image data in accordance with additional embodiments of the present technology.

FIGS. 11A and 11B are schematic representations of a vertebra of a patient in accordance with embodiments of the present technology.

FIG. 12 is a flow diagram of a process or method for updating depth information of a physical scene after the scene changes in accordance with embodiments of the present technology.

FIGS. 13A and 13B illustrate different views of the user interface of FIGS. 4A-4C including the overlay of preoperative plan information in accordance with embodiments of the present technology.

FIGS. 14A-14C are an axial, sagittal, and coronal cutaway view, respectively, of 3D image data illustrating the overlay of a 3D representation of an implant in accordance with embodiments of the present technology.

### DETAILED DESCRIPTION

Aspects of the present technology are directed generally to image guided-navigation systems (e.g., augmented-reality imaging systems, virtual-reality imaging systems, mediated-reality imaging systems), such as for use in surgical procedures, and associated methods. In several of the embodiments described below, for example, an imaging system includes (i) a camera array including a plurality of cameras configured to capture intraoperative image data (e.g., light field data and/or depth data) of a surgical scene and (ii) a processing device communicatively coupled to the camera array. The camera array can further include one or more trackers configured to track one or more tools (e.g., instruments) through the surgical scene. The processing device can be configured to synthesize/generate a three-dimensional (3D) virtual image corresponding to a virtual perspective of the scene in real-time or near-real-time based on the image data from at least a subset of the cameras. The processing device can output the 3D virtual image to a display device (e.g., a head-mounted display (HMD)) for viewing by a viewer, such as a surgeon or other operator of the imaging system. The imaging system is further configured to receive and/or store preoperative image data. The preoperative image data can be medical scan data (e.g., computerized tomography (CT) scan data) corresponding to a portion of a patient in the scene, such as a spine of a patient undergoing a spinal surgical procedure.

The processing device can register the preoperative image data to the intraoperative image data by, for example, registering/matching fiducial markers and/or other feature points visible in 3D data sets representing both the preoperative and intraoperative image data. The processing device can further display the preoperative image on the display device along with a representation of the tool. This can allow a user, such as a surgeon, to simultaneously view the underlying 3D anatomy of a patient undergoing an operation and the position of the tool relative to the 3D anatomy.

In some embodiments, the processing can display a cross-section of the preoperative image data based on the position of the tool and/or the view of the user (e.g., based on the position and orientation of an HMD worn by the user and/or a virtual camera generated by the imaging system). In some embodiments, the processing device is configured to calculate a distance (e.g., depth) between the tool and a surface of the preoperative image data. In some embodiments, the distance can be displayed on the display device and updated in real-time. In some embodiments, the display device can provide a visual indication when the distance is less than a predefined threshold to, for example, provide the user with an indication that the tool may breach the anatomy of a patient and/or has breached the anatomy of the patient.

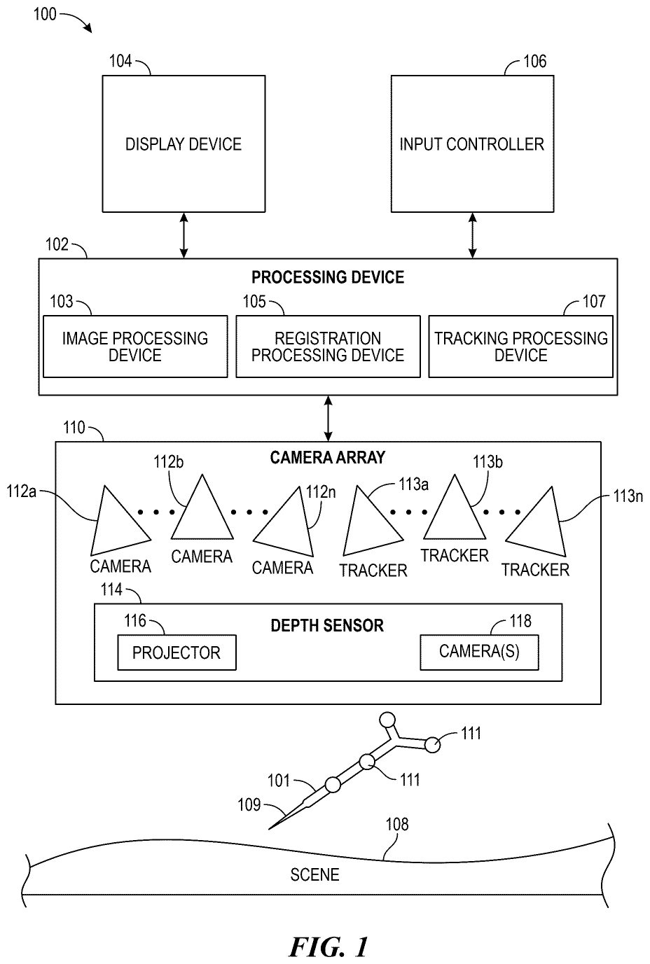

Specific details of several embodiments of the present technology are described herein with reference to FIGS. 1-14C. The present technology, however, can be practiced without some of these specific details. In some instances, well-known structures and techniques often associated with camera arrays, light field cameras, image reconstruction, registration processes, user interfaces, and the like have not been shown in detail so as not to obscure the present technology. The terminology used in the description presented below is intended to be interpreted in its broadest reasonable manner, even though it is being used in conjunction with a detailed description of certain specific embodiments of the disclosure. Certain terms can even be emphasized below; however, any terminology intended to be interpreted in any restricted manner will be overtly and specifically defined as such in this Detailed Description section.

Moreover, although frequently described in the context of displaying preoperative image data and/or intraoperative image data of a spinal surgical scene, the methods and systems of the present technology can be used to display image data of other types. For example, the systems and methods of the present technology can be used more generally to display any previously-captured image data of a scene to generate a mediated reality view of the scene including a fusion of the previously-captured data and real-time images.

The accompanying figures depict embodiments of the present technology and are not intended to be limiting of its scope. Depicted elements are not necessarily drawn to scale, and various elements can be arbitrarily enlarged to improve legibility. Component details can be abstracted in the figures to exclude details as such details are unnecessary for a complete understanding of how to make and use the present technology. Many of the details, dimensions, angles, and other features shown in the Figures are merely illustrative of particular embodiments of the disclosure. Accordingly, other embodiments can have other dimensions, angles, and features without departing from the spirit or scope of the present technology.

The headings provided herein are for convenience only and should not be construed as limiting the subject matter disclosed. To the extent any materials incorporated herein by reference conflict with the present disclosure, the present disclosure controls.

### I. Selected Embodiments of Imaging Systems

FIG. 1 is a schematic view of an imaging system 100 (“system 100”) in accordance with embodiments of the present technology. In some embodiments, the system 100 can be a synthetic augmented reality system, a virtual-reality imaging system, an augmented-reality imaging system, a mediated-reality imaging system, and/or a non-immersive computational imaging system. In the illustrated embodiment, the system 100 includes a processing device 102 that is communicatively coupled to one or more display devices 104, one or more input controllers 106, and a camera array 110. In other embodiments, the system 100 can comprise additional, fewer, or different components. In some embodiments, the system 100 can include some features that are generally similar or identical to those of the mediated-reality imaging systems disclosed in (i) U.S. patent application Ser. No. 16/586,375, titled “CAMERA ARRAY FOR A MEDIATED-REALITY SYSTEM,” and filed Sep. 27, 2019 and/or (ii) U.S. patent application Ser. No. 15/930,305, titled “METHODS AND SYSTEMS FOR IMAGING A SCENE, SUCH AS A MEDICAL SCENE, AND TRACKING OBJECTS WITHIN THE SCENE,” and filed May 12, 2020, each of which is incorporated herein by reference in its entirety.

In the illustrated embodiment, the camera array 110 includes a plurality of cameras 112 (identified individually as cameras 112a-112n; which can also be referred to as first cameras) that are each configured to capture images of a scene 108 from a different perspective (e.g., first image data). The scene 108 might include for example, a patient undergoing surgery or another medical procedure. In other embodiments, the scene 108 can be another type of scene. The camera array 110 further includes a plurality of dedicated object tracking hardware 113 (identified individually as trackers 113a-113n) configured to capture positional data of one more objects, such as an instrument 101 (e.g., a surgical instrument or tool) having a tip 109, to track the movement and/or orientation of the objects through/in the scene 108. In some embodiments, the cameras 112 and the trackers 113 are positioned at fixed locations and orientations (e.g., poses) relative to one another. For example, the cameras 112 and the trackers 113 can be structurally secured by/to a mounting structure (e.g., a frame) at predefined fixed locations and orientations. In some embodiments, the cameras 112 can be positioned such that neighboring cameras 112 share overlapping views of the scene 108. In general, the position of the cameras 112 can be selected to maximize clear and accurate capture of all or a selected portion of the scene 108. Likewise, the trackers 113 can be positioned such that neighboring trackers 113 share overlapping views of the scene 108. Therefore, all or a subset of the cameras 112 and the trackers 113 can have different extrinsic parameters, such as position and orientation.

In some embodiments, the cameras 112 in the camera array 110 are synchronized to capture images of the scene 108 simultaneously (within a threshold temporal error). In some embodiments, all or a subset of the cameras 112 can be light field, plenoptic, RGB, and/or hyperspectral cameras that are configured to capture information about the light field emanating from the scene 108 (e.g., information about the intensity of light rays in the scene 108 and also information about a direction the light rays are traveling through space). Therefore, in some embodiments the images captured by the cameras 112 can encode depth information representing a surface geometry of the scene 108. In some embodiments, the cameras 112 are substantially identical. In other embodiments, the cameras 112 can include multiple cameras of different types. For example, different subsets of the cameras 112 can have different intrinsic parameters such as focal length, sensor type, optical components, and the like. The cameras 112 can have charge-coupled device (CCD) and/or complementary metal-oxide semiconductor (CMOS) image sensors and associated optics. Such optics can include a variety of configurations including lensed or bare individual image sensors in combination with larger macro lenses, micro-lens arrays, prisms, and/or negative lenses. For example, the cameras 112 can be separate light field cameras each having their own image sensors and optics. In other embodiments, some or all of the cameras 112 can comprise separate microlenslets (e.g., lenslets, lenses, microlenses) of a microlens array (MLA) that share a common image sensor.

In some embodiments, the trackers 113 are imaging devices, such as infrared (IR) cameras that are each configured to capture images of the scene 108 from a different perspective compared to other ones of the trackers 113. Accordingly, the trackers 113 and the cameras 112 can have different spectral sensitives (e.g., infrared vs. visible wavelength). In some embodiments, the trackers 113 are configured to capture image data of a plurality of optical markers (e.g., fiducial markers, marker balls) in the scene 108, such as markers 111 coupled to the instrument 101.

In the illustrated embodiment, the camera array 110 further includes a depth sensor 114. In some embodiments, the depth sensor 114 includes (i) one or more projectors 116 configured to project a structured light pattern onto/into the scene 108 and (ii) one or more depth cameras 118 (which can also be referred to as second cameras) configured to capture second image data of the scene 108 including the structured light projected onto the scene 108 by the projector 116. The projector 116 and the depth cameras 118 can operate in the same wavelength and, in some embodiments, can operate in a wavelength different than the cameras 112. For example, the cameras 112 can capture the first image data in the visible spectrum, while the depth cameras 118 capture the second image data in the infrared spectrum. In some embodiments, the depth cameras 118 have a resolution that is less than a resolution of the cameras 112. For example, the depth cameras 118 can have a resolution that is less than 70%, 60%, 50%, 40%, 30%, or 20% of the resolution of the cameras 112. In other embodiments, the depth sensor 114 can include other types of dedicated depth detection hardware (e.g., a LiDAR detector) for determining the surface geometry of the scene 108. In other embodiments, the camera array 110 can omit the projector 116 and/or the depth cameras 118.

In the illustrated embodiment, the processing device 102 includes an image processing device 103 (e.g., an image processor, an image processing module, an image processing unit), a registration processing device 105 (e.g., a registration processor, a registration processing module, a registration processing unit), and a tracking processing device 107 (e.g., a tracking processor, a tracking processing module, a tracking processing unit). The image processing device 103 is configured to (i) receive the first image data captured by the cameras 112 (e.g., light field images, hyperspectral images, light field image data, RGB images) and depth information from the depth sensor 114 (e.g., the second image data captured by the depth cameras 118), and (ii) process the image data and depth information to synthesize (e.g., generate, reconstruct, render) a three-dimensional (3D) output image of the scene 108 corresponding to a virtual camera perspective. The output image can correspond to an approximation of an image of the scene 108 that would be captured by a camera placed at an arbitrary position and orientation corresponding to the virtual camera perspective. In some embodiments, the image processing device 103 is further configured to receive and/or store calibration data for the cameras 112 and/or the depth cameras 118 and to synthesize the output image based on the image data, the depth information, and/or the calibration data. More specifically, the depth information and calibration data can be used/combined with the images from the cameras 112 to synthesize the output image as a 3D (or stereoscopic 2D) rendering of the scene 108 as viewed from the virtual camera perspective. In some embodiments, the image processing device 103 can synthesize the output image using any of the methods disclosed in U.S. patent application Ser. No. 16/457,780, titled “SYNTHESIZING AN IMAGE FROM A VIRTUAL PERSPECTIVE USING PIXELS FROM A PHYSICAL IMAGER ARRAY WEIGHTED BASED ON DEPTH ERROR SENSITIVITY,” and filed Jun. 28, 2019, which is incorporated herein by reference in its entirety. In other embodiments, the image processing device 103 is configured to generate the virtual camera perspective based only on the images captured by the cameras 112—without utilizing depth information from the depth sensor 114. For example, the image processing device 103 can generate the virtual camera perspective by interpolating between the different images captured by one or more of the cameras 112.

The image processing device 103 can synthesize the output image from images captured by a subset (e.g., two or more) of the cameras 112 in the camera array 110, and does not necessarily utilize images from all of the cameras 112. For example, for a given virtual camera perspective, the processing device 102 can select a stereoscopic pair of images from two of the cameras 112 that are positioned and oriented to most closely match the virtual camera perspective. In some embodiments, the image processing device 103 (and/or the depth sensor 114) is configured to estimate a depth for each surface point of the scene 108 relative to a common origin and to generate a point cloud and/or a 3D mesh that represents the surface geometry of the scene 108. For example, in some embodiments the depth cameras 118 of the depth sensor 114 can detect the structured light projected onto the scene 108 by the projector 116 to estimate depth information of the scene 108. In some embodiments, the image processing device 103 can estimate depth from multiview image data from the cameras 112 using techniques such as light field correspondence, stereo block matching, photometric symmetry, correspondence, defocus, block matching, texture-assisted block matching, structured light, and the like, with or without utilizing information collected by the depth sensor 114. In other embodiments, depth may be acquired by a specialized set of the cameras 112 performing the aforementioned methods in another wavelength.

In some embodiments, the registration processing device 105 is configured to receive and/or store previously-captured image data, such as image data of a three-dimensional volume of a patient (3D image data). The image data can include, for example, computerized tomography (CT) scan data, magnetic resonance imaging (MRI) scan data, ultrasound images, fluoroscope images, and/or other medical or other image data. The registration processing device 105 is further configured to register the preoperative image data to the real-time images captured by the cameras 112 and/or the depth sensor 114 by, for example, determining one or more transforms/transformations/mappings between the two. The processing device 102 (e.g., the image processing device 103) can then apply the one or more transforms to the preoperative image data such that the preoperative image data can be aligned with (e.g., overlaid on) the output image of the scene 108 in real-time or near real time on a frame-by-frame basis, even as the virtual perspective changes. That is, the image processing device 103 can fuse the preoperative image data with the real-time output image of the scene 108 to present a mediated-reality view that enables, for example, a surgeon to simultaneously view a surgical site in the scene 108 and the underlying 3D anatomy of a patient undergoing an operation. In some embodiments, the registration processing device 105 can register the previously-captured image data to the real-time images by using any of the methods disclosed in U.S. patent application Ser. No. 17/140,885, titled “METHODS AND SYSTEMS FOR REGISTERING PREOPERATIVE IMAGE DATA TO INTRAOPERATIVE IMAGE DATA OF A SCENE, SUCH AS A SURGICAL SCENE,” and filed Jan. 4, 2021, which is incorporated herein by reference in its entirety.

In some embodiments, the tracking processing device 107 can process positional data captured by the trackers 113 to track objects (e.g., the instrument 101) within the vicinity of the scene 108. For example, the tracking processing device 107 can determine the position of the markers 111 in the 2D images captured by two or more of the trackers 113, and can compute the 3D position of the markers 111 via triangulation of the 2D positional data. More specifically, in some embodiments the trackers 113 include dedicated processing hardware for determining positional data from captured images, such as a centroid of the markers 111 in the captured images. The trackers 113 can then transmit the positional data to the tracking processing device 107 for determining the 3D position of the markers 111. In other embodiments, the tracking processing device 107 can receive the raw image data from the trackers 113. In a surgical application, for example, the tracked object may comprise a surgical instrument, an implant, a hand or arm of a physician or assistant, and/or another object having the markers 111 mounted thereto. In some embodiments, the processing device 102 can recognize the tracked object as being separate from the scene 108, and can apply a visual effect to the 3D output image to distinguish the tracked object by, for example, highlighting the object, labeling the object, and/or applying a transparency to the object.

In some embodiments, functions attributed to the processing device 102, the image processing device 103, the registration processing device 105, and/or the tracking processing device 107 can be practically implemented by two or more physical devices. For example, in some embodiments a synchronization controller (not shown) controls images displayed by the projector 116 and sends synchronization signals to the cameras 112 to ensure synchronization between the cameras 112 and the projector 116 to enable fast, multi-frame, multi-camera structured light scans. Additionally, such a synchronization controller can operate as a parameter server that stores hardware specific configurations such as parameters of the structured light scan, camera settings, and camera calibration data specific to the camera configuration of the camera array 110. The synchronization controller can be implemented in a separate physical device from a display controller that controls the display device 104, or the devices can be integrated together.

The processing device 102 can comprise a processor and a non-transitory computer-readable storage medium that stores instructions that when executed by the processor, carry out the functions attributed to the processing device 102 as described herein. Although not required, aspects and embodiments of the present technology can be described in the general context of computer-executable instructions, such as routines executed by a general-purpose computer, e.g., a server or personal computer. Those skilled in the relevant art will appreciate that the present technology can be practiced with other computer system configurations, including Internet appliances, hand-held devices, wearable computers, cellular or mobile phones, multi-processor systems, microprocessor-based or programmable consumer electronics, set-top boxes, network PCs, mini-computers, mainframe computers and the like. The present technology can be embodied in a special purpose computer or data processor that is specifically programmed, configured or constructed to perform one or more of the computer-executable instructions explained in detail below. Indeed, the term “computer” (and like terms), as used generally herein, refers to any of the above devices, as well as any data processor or any device capable of communicating with a network, including consumer electronic goods such as game devices, cameras, or other electronic devices having a processor and other components, e.g., network communication circuitry.

The present technology can also be practiced in distributed computing environments, where tasks or modules are performed by remote processing devices, which are linked through a communications network, such as a Local Area Network (“LAN”), Wide Area Network (“WAN”), or the Internet. In a distributed computing environment, program modules or sub-routines can be located in both local and remote memory storage devices. Aspects of the present technology described below can be stored or distributed on computer-readable media, including magnetic and optically readable and removable computer discs, stored as in chips (e.g., EEPROM or flash memory chips). Alternatively, aspects of the present technology can be distributed electronically over the Internet or over other networks (including wireless networks). Those skilled in the relevant art will recognize that portions of the present technology can reside on a server computer, while corresponding portions reside on a client computer. Data structures and transmission of data particular to aspects of the present technology are also encompassed within the scope of the present technology.

The virtual camera perspective is controlled by an input controller 106 that update the virtual camera perspective based on user driven changes to the camera's position and rotation. The output images corresponding to the virtual camera perspective can be outputted to the display device 104. In some embodiments, the image processing device 103 can vary the perspective, the depth of field (e.g., aperture), the focus plane, and/or another parameter of the virtual camera (e.g., based on an input from the input controller) to generate different 3D output images without physically moving the camera array 110. The display device 104 is configured to receive output images (e.g., the synthesized 3D rendering of the scene 108) and to display the output images for viewing by one or more viewers. In some embodiments, the processing device 102 can receive and process inputs from the input controller 106 and process the captured images from the camera array 110 to generate output images corresponding to the virtual perspective in substantially real-time as perceived by a viewer of the display device 104 (e.g., at least as fast as the frame rate of the camera array 110).

Additionally, the display device 104 can display a graphical representation on/in the image of the virtual perspective of any (i) tracked objects within the scene 108 (e.g., a surgical tool) and/or (ii) registered or unregistered preoperative image data. That is, for example, the system 100 (e.g., via the display device 104) can blend augmented data into the scene 108 by overlaying and aligning information on top of “passthrough” images of the scene 108 captured by the cameras 112. Moreover, the system 100 can create a modulated reality experience where the scene 108 is reconstructed using light field image date of the scene 108 captured by the cameras 112, and where instruments are virtually represented in the reconstructed scene via information from the trackers 113. Additionally or alternatively, the system 100 can remove the original scene 108 and completely replace it with a registered and representative arrangement of the preoperatively captured image data, thereby removing information in the scene 108 that is not pertinent to a user's task.

The display device 104 can comprise, for example, a head-mounted display device, a monitor, a computer display, and/or another display device. In some embodiments, the input controller 106 and the display device 104 are integrated into a head-mounted display device and the input controller 106 comprises a motion sensor that detects position and orientation of the head-mounted display device. The virtual camera perspective can then be derived to correspond to the position and orientation of the head-mounted display device 104 in the same reference frame and at the calculated depth (e.g., as calculated by the depth sensor 114) such that the virtual perspective corresponds to a perspective that would be seen by a viewer wearing the head-mounted display device 104. Thus, in such embodiments the head-mounted display device 104 can provide a real-time rendering of the scene 108 as it would be seen by an observer without the head-mounted display device 104. Alternatively, the input controller 106 can comprise a user-controlled control device (e.g., a mouse, pointing device, handheld controller, gesture recognition controller, etc.) that enables a viewer to manually control the virtual perspective displayed by the display device 104.

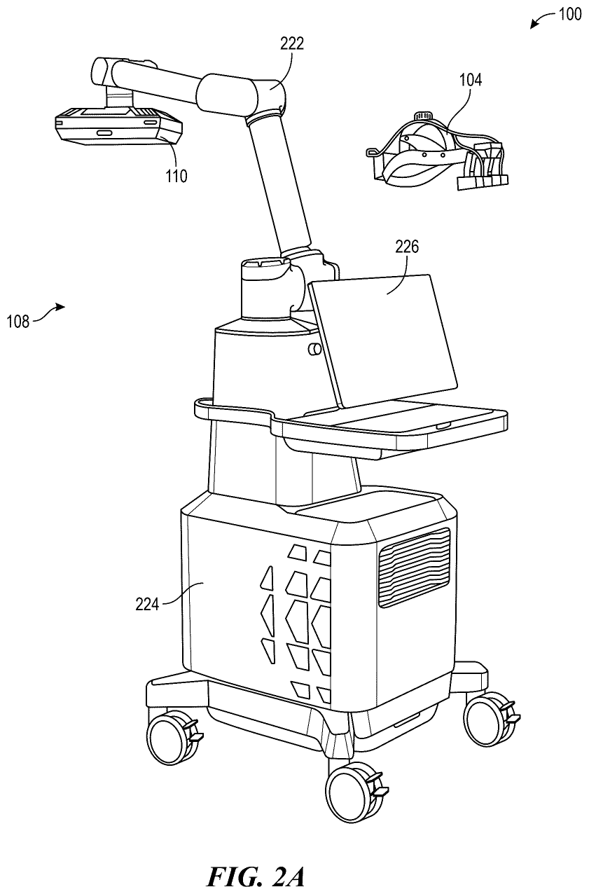

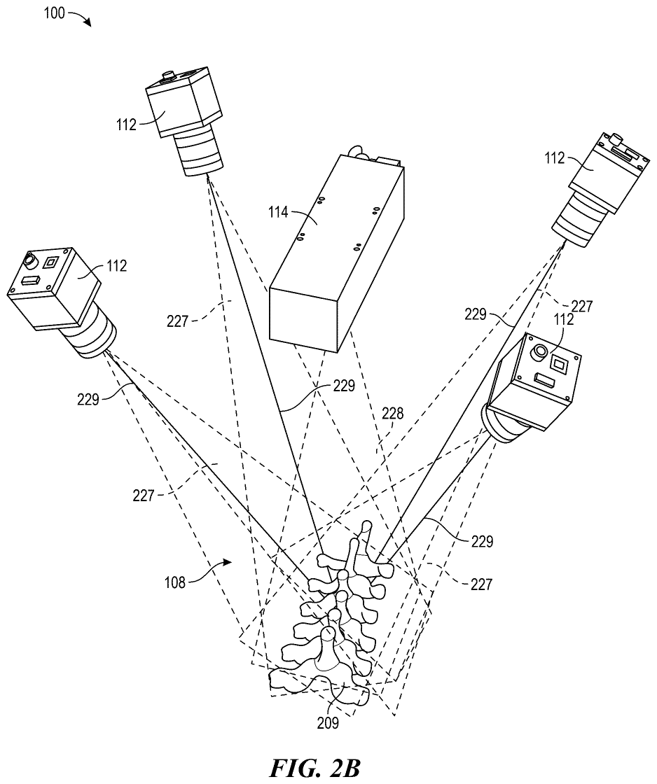

FIG. 2A is a perspective view of a surgical environment employing the system 100 for a surgical application in accordance with embodiments of the present technology. In the illustrated embodiment, the camera array 110 is positioned over the scene 108 (e.g., a surgical site) and supported/positioned via a movable arm 222 that is operably coupled to a workstation 224. In some embodiments, the arm 222 can be manually moved to position the camera array 110 while, in other embodiments, the arm 222 can be robotically controlled in response to the input controller 106 (FIG. 1) and/or another controller. In the illustrated embodiment, the display device 104 is a head-mounted display device (e.g., a virtual reality headset, augmented reality headset, etc.). The workstation 224 can include a computer to control various functions of the processing device 102, the display device 104, the input controller 106, the camera array 110, and/or other components of the system 100 shown in FIG. 1. Accordingly, in some embodiments the processing device 102 and the input controller 106 are each integrated in the workstation 224. In some embodiments, the workstation 224 includes a secondary display 226 that can display a user interface for performing various configuration functions, a mirrored image of the display on the display device 104, and/or other useful visual images/indications. In other embodiments, the system 100 can include more or fewer display devices. For example, in addition to the display device 104 and the secondary display 226, the system 100 can include another display (e.g., a medical grade computer monitor) visible to the user wearing the display device 104.

FIG. 2B is an isometric view of a portion of the system 100 illustrating four of the cameras 112 in accordance with embodiments of the present technology. Other components of the system 100 (e.g., other portions of the camera array 110, the processing device 102, etc.) are not shown in FIG. 2B for the sake of clarity. In the illustrated embodiment, each of the cameras 112 has a field of view 227 and a focal axis 229. Likewise, the depth sensor 114 can have a field of view 228 aligned with a portion of the scene 108. The cameras 112 can be oriented such that the fields of view 227 are aligned with a portion of the scene 108 and at least partially overlap one another to together define an imaging volume. In some embodiments, some or all of the fields of view 227, 228 at least partially overlap. For example, in the illustrated embodiment the fields of view 227, 228 converge toward a common measurement volume including a portion of a spine 209 of a patient (e.g., a human patient) located in/at the scene 108. In some embodiments, the cameras 112 are further oriented such that the focal axes 229 converge to a common point in the scene 108. In some aspects of the present technology, the convergence/alignment of the focal axes 229 can generally maximize disparity measurements between the cameras 112. In some embodiments, the cameras 112 and the depth sensor 114 are fixedly positioned relative to one another (e.g., rigidly mounted to a common frame) such that the positions of the cameras 112 and the depth sensor 114 relative to one another is known and/or can be readily determined via a calibration process. In other embodiments, the system 100 can include a different number of the cameras 112 and/or the cameras 112 can be positioned differently relative to another. In some embodiments, the camera array 110 can be moved (e.g., via the arm 222 of FIG. 2) to move the fields of view 227, 228 to, for example, scan the spine 209.

Referring to FIGS. 1-2B together, in some aspects of the present technology the system 100 can generate a digitized view of the scene 108 that provides a user (e.g., a surgeon) with increased “volumetric intelligence” of the scene 108. For example, the digitized scene 108 can be presented to the user from the perspective, orientation, and/or viewpoint of their eyes such that they effectively view the scene 108 as though they were not viewing the digitized image (e.g., as though they were not wearing the head-mounted display 104). However, the digitized scene 108 permits the user to digitally rotate, zoom, crop, or otherwise enhance their view to, for example, facilitate a surgical workflow. Likewise, initial image data, such as CT scans, can be registered to and overlaid over the image of the scene 108 to allow a surgeon to view these data sets together. Such a fused view can allow the surgeon to visualize aspects of a surgical site that may be obscured in the physical scene 108—such as regions of bone and/or tissue that have not been surgically exposed.

### II. Selected Embodiments of User Interfaces and Associated Methods and Systems

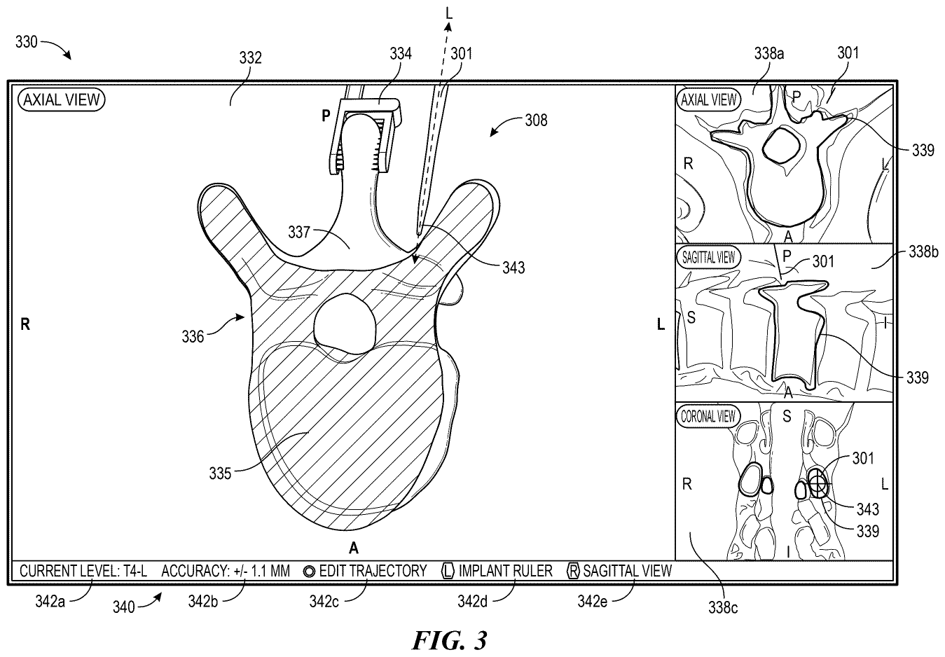

FIG. 3 illustrates a user interface (e.g., a display) 330 visible to a user of the system 100 via the display device 104 (e.g., a head-mounted display device) and/or the secondary display 226 in accordance with embodiments of the present technology. In the illustrated embodiment, the user interface 330 includes a primary viewport or panel 332 displaying a 3D view (“3D view 332”) of a physical scene 308, such as a surgical scene. In the illustrated embodiment, the physical scene 308 includes a dynamic reference frame (DRF) marker 334 and an instrument 301 (e.g., a tool, object), such as a surgical instrument. With additional reference to FIGS. 1-2B, the camera array 110 can track and/or image the DRF marker 334 and the instrument 301. As described in further detail below, the position of the DRF marker 334 can be used to dynamically update a registration between the physical scene 308 and previously-captured 3D image data. In other embodiments, registration can be continuously maintained using other suitable registration methods without relying on the DRF marker 334. The image processing device 103 can render 3D representations of the instrument 301 on the user interface 330 in the 3D view 332 and, in some embodiments, can also display a 3D representation of the DRF marker 334. Accordingly, the DRF marker 334 and/or the instrument 301 can be moved through the scene 308 and are represented and updated in real-time or near real-time on the user interface 330 in the 3D view 332 as 3D objects. In some embodiments, the 3D view 332 can include other image data captured by the cameras 112 and processed by the image processing device 103. For example, where the scene 308 is a surgical scene, the 3D view 332 can include/display an output image (e.g., including a 3D representation of a patient's spine) synthesized by the image processing device 103 from images captured by two or more of the cameras 112 in the camera array 110. That is, the 3D view 332 can display additional information about the physical scene 308 captured by the camera array 110.

The 3D view 332 can further include/display previously-captured 3D image data 336 that is registered to the physical scene 308. In some embodiments, the previously-captured 3D image data 336 (“3D image data 336”; e.g., initial image data) is preoperative image data. For example, in the illustrated embodiment the 3D image data 336 includes 3D geometric and/or volumetric data of a patient's vertebrae, such as computed tomography (CT) scan data, magnetic resonance imaging (MRI) scan data, ultrasound image data, fluoroscopic image data, and/or other medical or other image data. In some embodiments, the previously-captured 3D image data 336 can be captured intraoperatively. For example, the previously-captured 3D image data 336 can comprise 2D or 3D X-ray images, fluoroscopic images, CT images, MRI images, etc., and combinations thereof, captured of the patient within an operating room. In some embodiments, the previously-captured 3D image data 336 comprises a point cloud, three-dimensional (3D) mesh, and/or another 3D data set. In some embodiments, the previously-captured 3D image data 336 comprises segmented 3D CT scan data of some or all of the spine of the patient (e.g., segmented on a per-vertebra basis).

As described in greater detail below, in the illustrated embodiment the 3D image data 336 is displayed in cross-section. The 3D image data 336 can be registered to the physical scene 308 using a suitable registration process. In some embodiments, the 3D image data 336 can be registered to the physical scene 308 by comparing corresponding points in both the 3D image data 336 and the physical scene 308. For example, the user can touch the instrument 301 to points in the physical scene 308 corresponding to identified points in the 3D image data 336, such as pre-planned screw entry points on a patient's vertebra. The system 100 can then generate a registration transform between the 3D image data 336 and the physical scene 308 by comparing the points.

In some embodiments, the 3D image data 336 can be further registered to the physical scene 308 using the DRF marker 334. With additional reference to FIGS. 1-2B, for example, the registration process can include attaching the DRF marker 334 to the patient's vertebra, locating the DRF marker 334 (e.g., marker balls attached thereto) using the trackers 113, and generating an additional registration transform between the 3D image data 336 and the physical scene 308 based on the position and orientation of the DRF marker 334 in the physical scene 308. The DRF marker 334 can therefore be used to update the registration when the physical scene 308 changes, such as when the user pushes the instrument 301 against the vertebra. Accordingly, after registration, the 3D image data 336 is aligned with the physical scene 308 in the 3D view 332. That is, for example, the instrument 301, the DRF marker 334, and 3D image data 336 are represented in the 3D view 332 as the instrument 301, the DRF marker 334, and the physical object (e.g., the patient's vertebrae) corresponding to the 3D image data 336 exist in the physical scene 308.

In the illustrated embodiment, the user interface 330 further includes a plurality of additional secondary viewports or panels 338 each displaying a different 2D view (“first through third 2D views 338a-c,” respectively). In the illustrated embodiment, the primary viewport 332 is larger than the secondary viewports 338 while, in other embodiments, the viewports 332, 338 can have different sizes and/or relative positions along the user interface 330. In some embodiments, the 3D image data 336 can be a segmented portion of a 3D model generated from multiple 2D images. For example, the 3D model can be a volumetric representation of a patient's spine and the 3D image data 336 can be a segmented 3D geometry of the spine that removes extraneous information or noise. Accordingly, the 2D views 338 can each be a 2D image corresponding to the 3D image data 336. For example, in the illustrated embodiment the first 2D view 338a is a 2D axial CT view of the patient's spine, the second 2D view 338b is a 2D sagittal CT view of the patient's spine, and the third 2D view 338c is a 2D coronal CT view of the patient's spine. In some aspects of the present technology, the 2D views 338 allow the user to triangulate a spatial representation of the data in a manner that provides a clear understanding of the horizontal, vertical, and depth positions of a point of interest in the data (e.g., a tip 343 of the instrument 301).

In some embodiments, the 2D views 338 can each include an outline 339 around a portion of the 2D image corresponding to the segmented 3D image data 336. That is, for example, the outlines 339 can extend around an individual vertebra shown in the 2D views 338 that corresponds to the segmented 3D image of the patient's vertebrae shown in the 3D view 332. In some embodiments, the instrument 301 can also be shown in the 2D views 338. For example, the tip 343 of the instrument 301 is represented as a cross-hair in the third 2D view 338. In some embodiments, the visual representation of the tip 343 can be more relevant to the user in the 2D coronal view of the vertebra, where a projection off the tip 343 of the instrument 301 can be difficult for the user to see. In other embodiments, the user interface 330 can include more, fewer, and/or different views. For example, with additional reference to FIGS. 1 and 2A, the 3D view 332 and the 2D views 338 can each be displayed on the secondary display 226, while only the 3D view 332 is presented on the display device 104 (e.g., a head-mounted display).

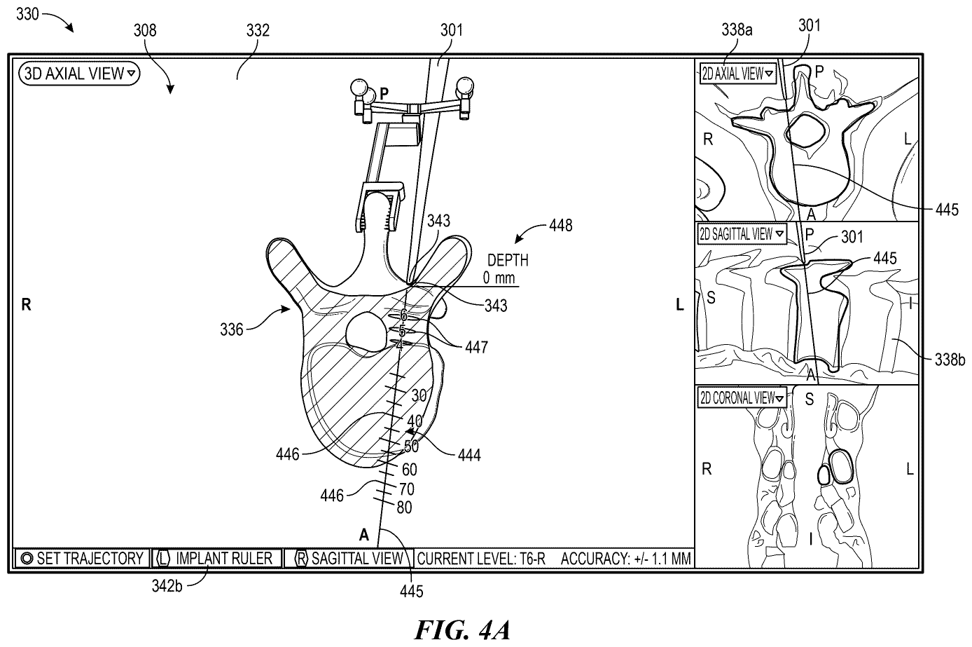

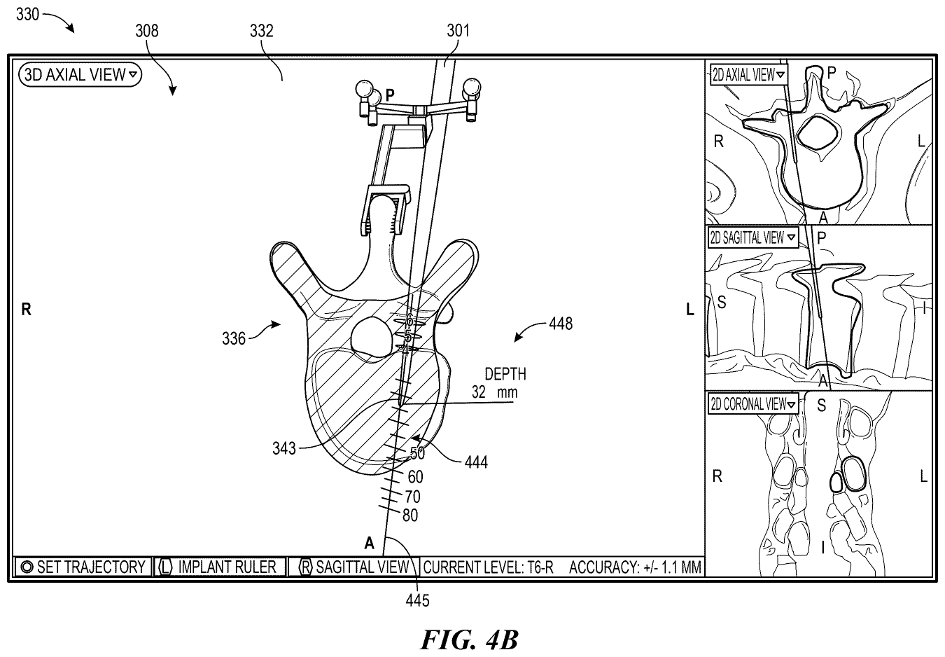

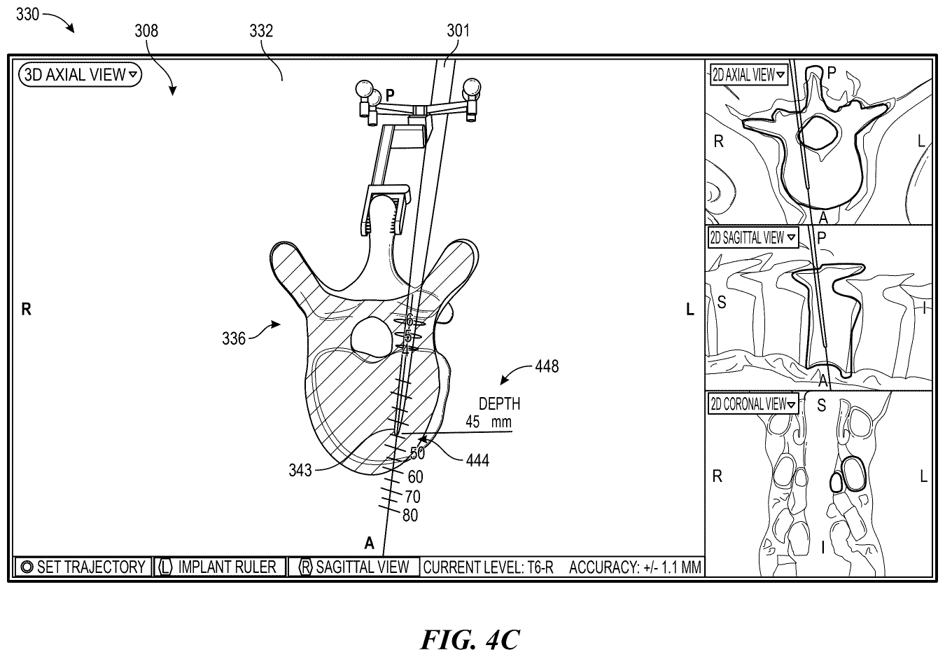

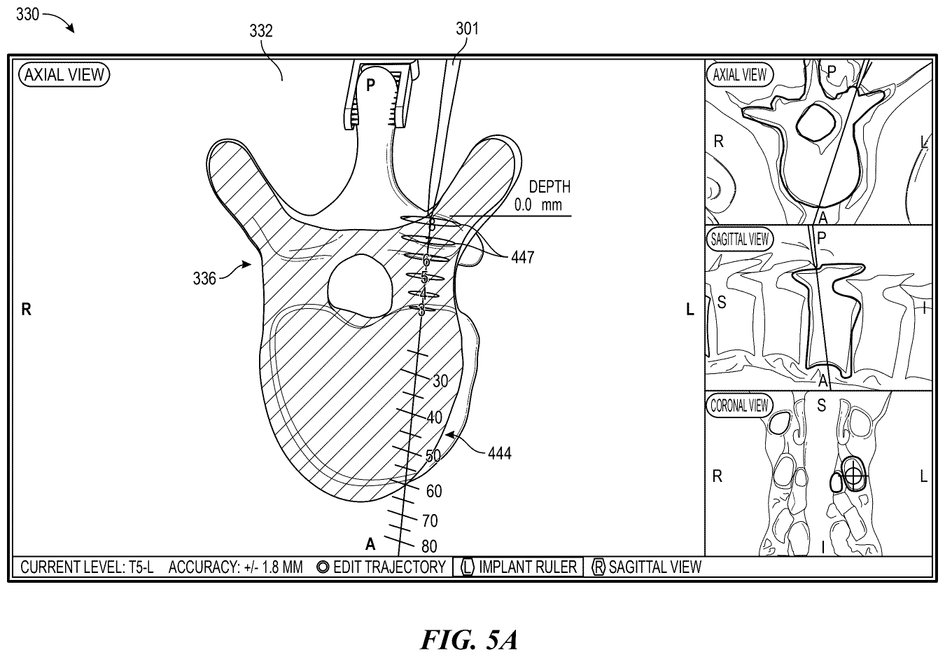

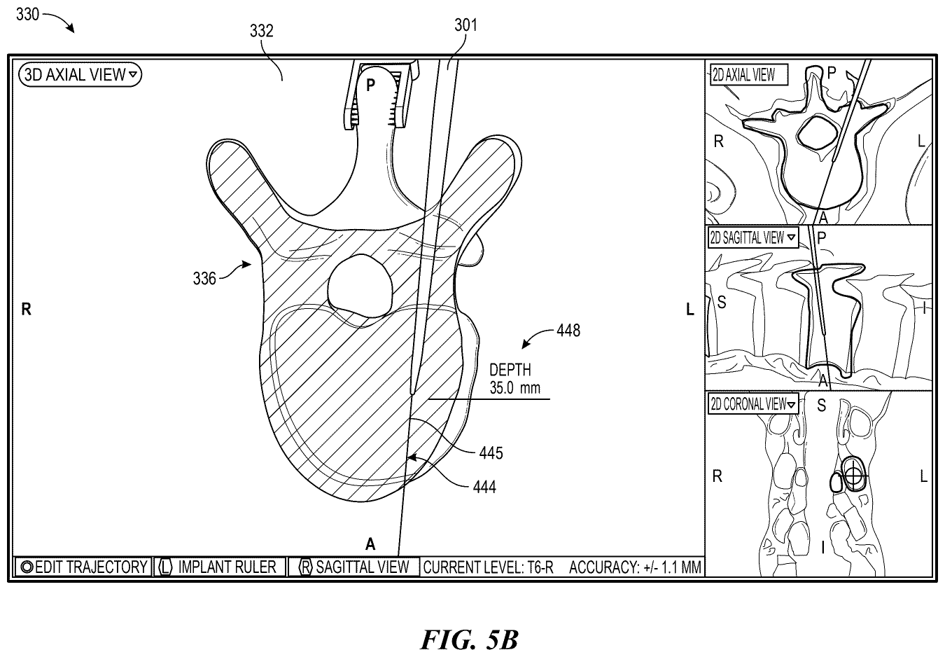

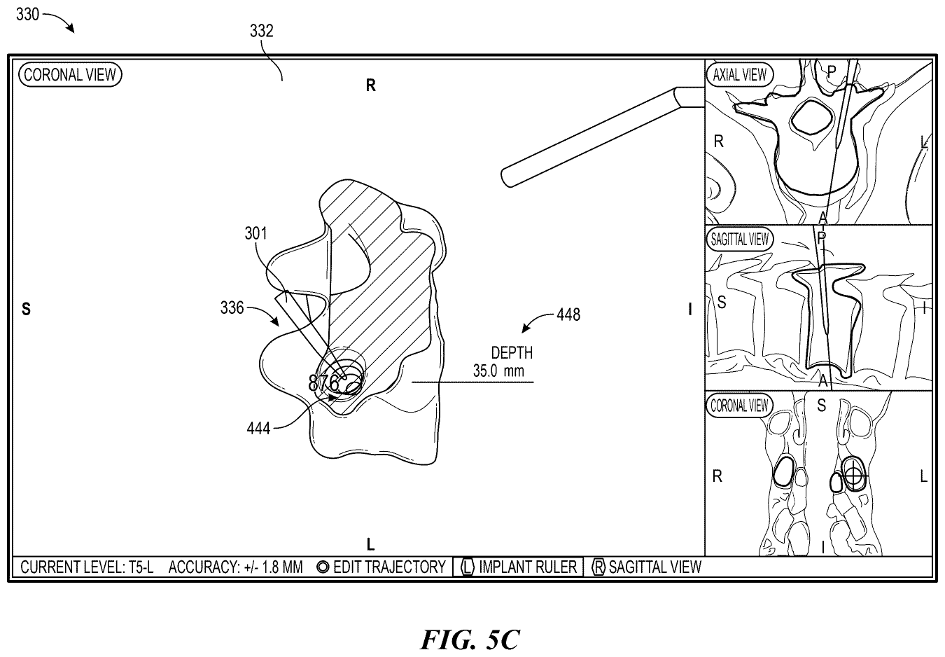

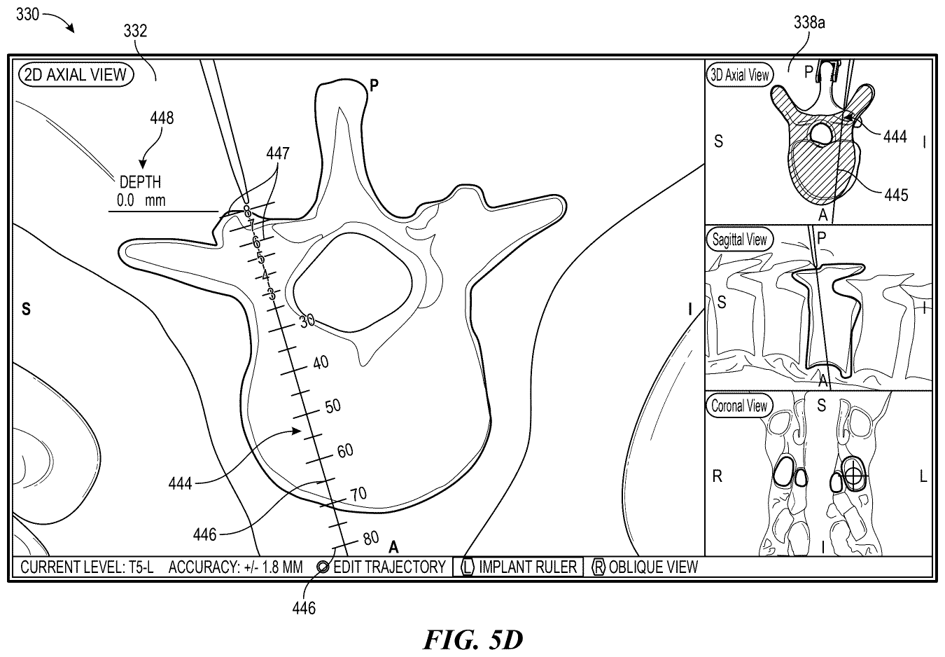

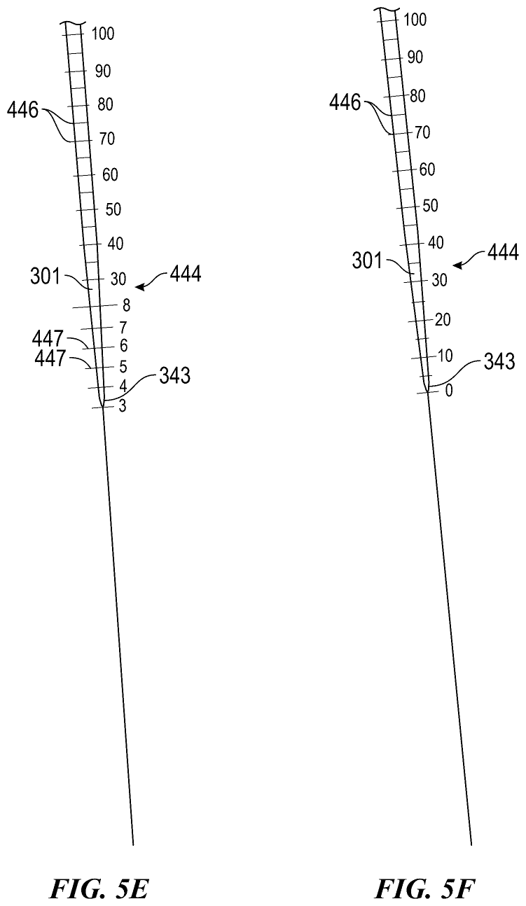

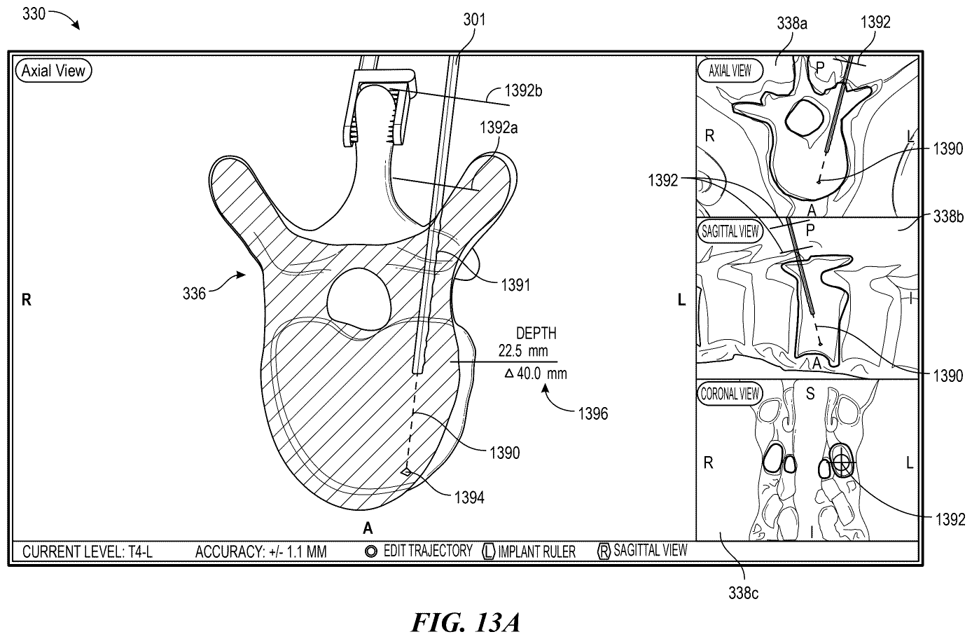

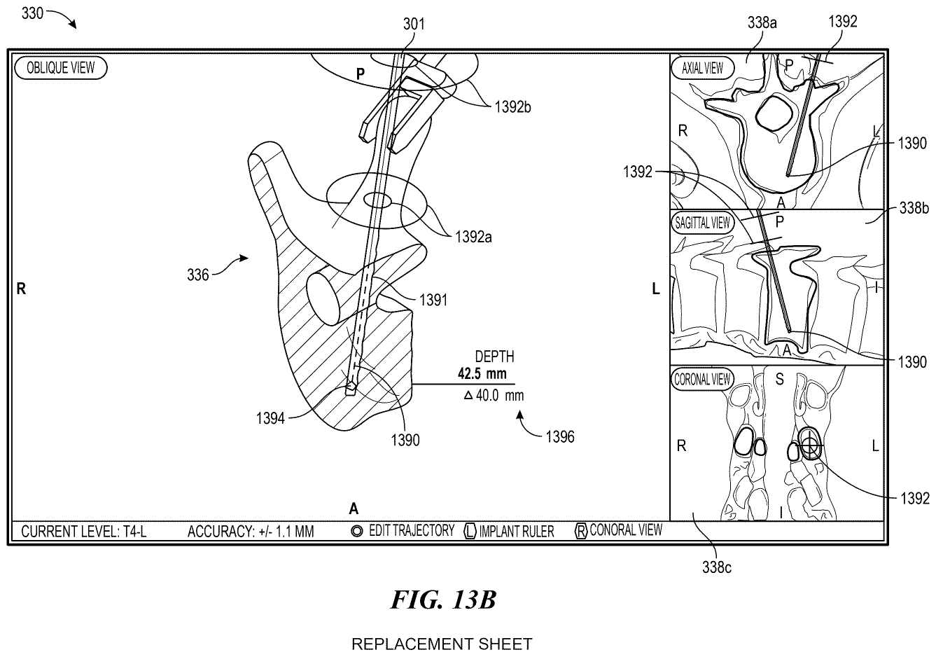

In some embodiments, the user interface 330 can include an information and/or options bar 340 including a plurality of icons 342 (identified individually as first through fifth icons 342a-e, respectively). In the illustrated embodiment, the third icon 342c displays an “edit trajectory” option, the fourth icon 342d displays a “ruler” option, and the fifth icon 342e displays a “view” option. In some embodiments, a user can provide a user input (e.g., a depression of a foot pedal, a touch on a touch screen, a head movement, a mouse click, and so on) to the third through fifth icons 342c-e to trigger their associated functionality. For example, a user input to the third icon 342c can cause a trajectory to be superimposed on the 3D view 332 and/or the 2D views 338, such as a pre-planned trajectory for an implant (e.g., a screw) or tool relative to the 3D image data 336 as described in detail below with reference to FIGS. 13A and 13B, a projected trajectory from the instrument 301, and so on. A user input to the fourth icon 342d can cause a ruler to be superimposed on the 3D view 332 and/or the 2D views 338 as described in detail below with reference to FIGS. 4A-5F. Likewise, a user input to the fifth icon 342e can change the perspective of the 3D view 332 (e.g., from an axial view to a sagittal view and so on) and/or swap the views between the various viewports 332, 338. In the illustrated embodiment, the first icon 342a displays information about the 3D image data 336, such as a vertebral level corresponding to the displayed segmented vertebra. The second icon 342b can display information about the registration of the 3D image data 336 to the physical scene 308, such as an accuracy measured in millimeters. In other embodiments, the information and/or options bar 340 can include icons displaying other types of information or triggering other functionality.

FIGS. 4A-4C illustrate the user interface 330 of FIG. 3 including the display of a ruler 444 in the 3D view 332 in accordance with embodiments of the present technology. More specifically, FIGS. 4A-4C illustrate the ruler 444 during different stages of a surgical procedure using the instrument 301, such as a procedure to implant a screw (e.g., a pedicle screw) in a vertebra. Accordingly, in some embodiments the instrument 301 can be a drill, screw driver, and/or the like. Although described in the context of spinal surgery, the ruler 444 can be implemented and displayed on the user interface 330 during other procedures.

Referring first to FIG. 4A, the display of the ruler 444 can be triggered via, for example, a user input associated with the second icon 342b. In some embodiments, the user can select/initialize the position of the ruler 444, by for example, positioning a tip 343 of the instrument 301 relative to the 3D image data 336 at an initialization point (e.g., at an entry point, a starting point). For example, the user can first position the instrument 301 against the physical vertebra in the physical scene 308 and then trigger the second icon 342b to initialize and locate the ruler 444 relative to the 3D image data 336, such as to extend from the tip 343 of the instrument 301. In some embodiments, the position of the tip 343 when the ruler 444 is initialized can be at a known distance, such as a zero distance, relative to the surface of the 3D image data 336. That is, the initialization point can be on the surface of the 3D image data 336. In other embodiments, the initialization point can be below or above the surface of the 3D image data 336.

In the illustrated embodiment, the ruler 444 includes a longitudinal axis 445, a plurality of depth indicators 446 aligned along the longitudinal axis 445, and a plurality of width indicators 447 aligned along the longitudinal axis 445. In some embodiments, the longitudinal axis 445 can be aligned with a longitudinal axis of the instrument 301 and can be initiated at a point on the surface of the 3D image data 336 corresponding to the position of the tip 343 of the instrument 301 at the initialization point. In other embodiments, the position and orientation of the longitudinal axis 445 can be manually or automatically selected without using the instrument 301. For example, the longitudinal axis 445 can be selected based on a pre-planned (e.g., preoperative) plan for the placement of a screw or other implant.

The depth indicators 446 can be hash marks or other indicators spaced along the longitudinal axis 445 that indicate a depth from the initialization point, such as a depth from the surface of the 3D image data 336. In some aspects of the present technology, the depth indicators 446 indicate a depth from the surface of the 3D image data 336 rather than from the position of the tip 343 of the instrument 301. The width indicators 447 can be concentric 3D circles or other indicators spaced along the longitudinal axis 445 and, in some embodiments, can be positioned closer to the surface starting point of the longitudinal axis 445 than the depth indicators 446. In some embodiments, the width indicators 447 can correspond to different widths of different screws or other implants to enable the user to visualize the size of a potential screw or implant relative to the actual size and anatomy of the vertebra represented by the 3D image data 336. The scale of the measurements provided by the ruler 444 can be based on scale information incorporated in the 3D image data 336.

In some embodiments, the ruler 444 can further include a depth readout 448 indicating a depth (e.g., a distance) of the tip 343 of the instrument 301 relative to the surface initialization point (e.g., starting point, tool entry point) where the ruler 444 was selected/initialized. In the illustrated embodiment, because the tip 343 of the instrument 301 is positioned at the surface starting point (e.g., on the surface of the vertebra), the depth readout 448 indicates a zero depth (e.g., “0 mm”).

In some embodiments, the instrument 301 and/or all or a portion of the ruler 444 can be displayed in one or more of the 2D views 338. In the illustrated embodiment, for example, the instrument 301 and the longitudinal axis 445 of the ruler 444 are displayed in the first 2D view 338a and in the second 2D view 338 b. Accordingly, in some aspects of the present technology, the system 100 can selectively display more or fewer components of the ruler 444 (e.g., more or less detail) based on the relative sizes of the 3D view 332 and the 2D views 338 on the user interface 330 to provide a desired amount of information to the user without cluttering any individual view and/or rendering the view unreadable.

FIG. 4B illustrates the user interface 330 after the instrument 301 has been partially inserted into the vertebra, and FIG. 4C illustrates the user interface 330 after the instrument 301 has been further inserted into the vertebra. Referring to FIGS. 4B and 4C together, after initialization of the ruler 444 as shown in FIG. 4A, the instrument 301 can move relative to the ruler 444 which can remain stationary (e.g., fixed in position) relative to the 3D image data 336. That is the position of the ruler 444 can be locked relative the 3D image data 336. As the depth of the tip 343 of the instrument 301 increases relative to the surface of the 3D image data 336-and the corresponding physical surface of the vertebra in the physical scene 308 registered to the 3D image data 336—the depth readout 448 can display the real-time depth of the tip 343 of the instrument 301 (e.g., “32 mm” in FIG. 4B and “45 mm” in FIG. 4C) relative to the surface of the vertebra. Accordingly, in some aspects of the present technology the ruler 444 provides real-time feedback to the user on the user interface 330 of the depth of the instrument 301 relative to the surface of the 3D image data 336 and the corresponding physical anatomy in the physical scene 308.

In some embodiments, in addition to or instead of locking the position of the ruler 444, the user interface 330 can display another visual representation of the instrument 301 relative to the 3D image data 336—such as at a position pre-selected during a preoperative planning procedure or selected in real-time during a procedure. In some aspects of the present technology, this can allow the user to visualize the desired position for the instrument 301 such that they can attempt to maintain alignment of the instrument 301 to the displayed visual representation during a procedure (e.g., as the user applies pressure with the instrument 301).

Accordingly, in some aspects of the present technology the ruler 444 and the depth readout 448 can assist the user with navigating the preoperatively acquired 3D image data 336—which is registered to the physical scene 308—in a way that supports high precision navigation of the tracked instrument 301. Additionally, the presentation of such visuals for assisting the user can be obscured or revealed based on the size of the visuals, the level of noise present in the scene 308 (e.g., near the tip 343 of the instrument 301), and/or based on other factors to provide a helpful and uncluttered presentation to the user.

For example, in some embodiments the ruler 444 can include more or fewer information indicators. FIG. 5A, for example, illustrates the ruler 444 with additional width indicators 447 (e.g., six instead of the three shown in FIG. 4A) before entry of the instrument 301 into the vertebra. In some embodiments, after entry of the instrument 301 into the vertebra the number of the width indicators 447 can be reduced to represent only relevant diameters selected by the user or determined based on the geometry of the 3D image data 336. For example, only the top three best fit width indicators 447 can be shown as illustrated in FIGS. 4B and 4C. FIG. 5B further illustrates the ruler 444 with the depth indicators 446 and the width indicators 447 omitted entirely. Thus, by selectively reducing the number of visual displays (e.g., the width indicators 447), the system 100 can reduce the clutter on the user interface 330 to, for example, help increase the focus of the user. In some embodiments, the number of visual displays presenting information to the user can be varied based on the position of the instrument 301 relative to the vertebra and/or a size or other dimension of the vertebra. For example, the width indicators 447 can be omitted based on the measured dimensions of the vertebra along a planned and/or projected trajectory of the instrument 301 relative to the 3D image data 336—such as by obscuring/removing dimensions that would not fit the geometry of the scene 308.

As described in detail above, the user interface 330 can change the perspective/orientation of the 3D view 332 in response to, for example, a user input (e.g., to the third icon 342c shown in FIG. 3). FIG. 5C, for example, illustrates the 3D view 332 from a different perspective than that shown in FIGS. 4A-4C. In the illustrated embodiment, the 3D view 332 provides a coronal (e.g., top down) view of the 3D image data 336 and the ruler 444. Likewise, FIG. 5D illustrates the user interface 330 with the first, primary viewport 332 displaying the 2D axial view of the vertebra and the secondary viewport 338a displaying the 3D view of the vertebra. In the illustrated embodiment, the ruler 444 is displayed on the 2D axial view including the depth indicators 446, the width indicators 447, and the depth readout 448, while the ruler 444 is displayed with less visual information (e.g., only the longitudinal axis 445) in the 3D view in the viewport 338a. Accordingly, the system 100 can vary the amount of visual information displayed on the user interface 330 based on the particular viewports 332, 338 (e.g., their relative sizes).

Each of FIGS. 4-5D illustrate the ruler 444 extending from the tip 343 of the instrument 301 away from the instrument 301. In other embodiments, the ruler 444 can extend in the opposite direction along an axis (e.g., shaft) of the instrument 301, such as a longitudinal axis L shown in FIG. 3. FIGS. 5E and 5F, for example, are views of the ruler 444 and the instrument 301 of FIGS. 4A-5D in accordance with additional embodiments of the present technology. Referring to FIGS. 5E and 5F together, the ruler 444 is reverse projected along a shaft of the instrument 301 and includes depth indicators 446 along the shaft of the instrument 301 starting from the tip 334. As shown in FIG. 5E, the ruler 444 can optionally include the width indicators 447 extending about the instrument 301. In some aspects of the present technology, the ruler 444 can be displayed along the shaft of the instrument 301 as shown in FIGS. 5E and 5F when the instrument 301 is positioned within the 3D image data 336 (FIGS. 3-4C; e.g., within bone of the patient) to provide a depth measurement from the tip 343 of the instrument 301 to, for example, a surface of the 3D image data 336.

Referring again to FIG. 3, in some embodiments the system 100 can calculate and display in real-time or near real-time a distance between the tip 343 of the instrument 301 and the 3D image data 336 along the longitudinal axis L of the instrument 301. For example, the system 100 can calculate the distance between the tip 343 and an intersection point of the 3D image data 336 along the longitudinal axis L. Such a distance could be, for example, the distance from the tip 343 along the longitudinal axis L to the exterior surface of the 3D image data 336 (e.g., the exterior surface of a cortical layer of a vertebra), which can inform the user of the distance until the tip 343 of the instrument 301 will touch the surface. Alternatively, for example, the distance can be the distance from the tip 343 along the longitudinal axis L to the interior surface of the 3D image data 336 (e.g., the interior surface of the cortical layer of the vertebra), which can inform the user of the distance until the instrument 301 breaches the vertebra.

In other embodiments, the distance can be calculated from another location on/relative to the instrument 301. For example, where the instrument 301 is a driver or other implement configured to interface/connect with an implant, the distance can be calculated from the tip of the implant rather than the instrument 301. In some embodiments, the size of the implant is known from a surgical plan, determined via a user input (e.g., a technician specifying a width and length of the implant), and/or can be determined via images from the camera array 110. Based on the known or determined size of the implant, the tip of the implant can be determined based on the known/determined size of the implant and the position of the tip 343 of the instrument 301 that is configured to be coupled to the implant.

Accordingly, in some aspects of the present technology the system 100 can effectively provide a virtual real-time measuring tape from the tip 343 to an intersection point with the 3D image data 336. The 3D image data 336 and corresponding intersection point can correspond to specific tissue types (e.g., skin, never, muscle, bone, etc.) or other kinds of objects (e.g., wood, metal, etc.). For example, during a surgical procedure on a bone wherein the 3D image data 336 corresponds to the bone, the system 100 can calculate the distance between the tip 343 and the 3D image data 336 of the bone during a percutaneous procedure when the instrument 301 is touched to the skin of the patient to provide an indication of the distance from the current location of the tip 343 on the skin to the bone. In some embodiments, the user can move the tip 343 across the skin to find a shorter trajectory to the bone through the skin. Similarly, where the 3D image data 336 corresponds to skin, the system 100 can calculate the distance between the tip 343 and the 3D image data 336 of the skin as the instrument 301 approaches the skin of the patient.

Referring to FIGS. 3-4C together, the 3D image data 336 is displayed as cutaway or sliced along a plane extending parallel to the page. In the illustrated embodiment, for example, the 3D image data 336 is sliced such that an inner surface 335 (FIG. 3; shown as gray) of the displayed vertebra is visible behind the selected plane along with a portion of an outer surface 337 (FIG. 3; shown as blue) of the displayed vertebra. In some embodiments, the 3D image data 336 includes volumetric data representing a “shell” of the vertebra such that the inner surface 335 includes detail about the physical geometry (e.g., depth, contours) of the vertebra. In some embodiments, the position/depth of the slicing plane (e.g., along an axis extending into the page) can be selected to correspond to the position of the tip 343 of the instrument 301. In some embodiments, the slicing plane can move together with the tip 343 as the instrument 301 moves while, in other embodiments, the slicing plane can remain aligned with the point on the surface of the 3D image data 336 corresponding to the position of the tip 343 of the instrument 301 in FIG. 4A where the ruler 444 is initiated. That is, the slicing plane can remain fixed in position relative to the entry point of the instrument 301 (e.g., orthogonal thereto) or can move along with the instrument 301.

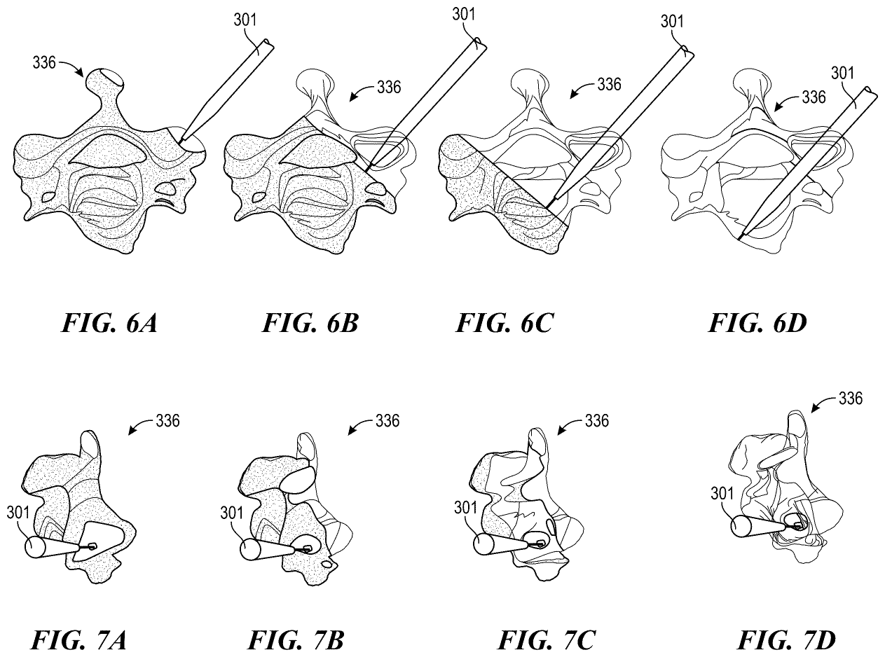

More specifically, for example, FIGS. 6A-6D illustrate the 3D image data 336 and the instrument 301 approaching, entering, and moving through the 3D image data 336 (and registered physical vertebra) in accordance with embodiments of the present technology. Referring to FIGS. 6A-6D, the 3D image data 336 is only partially or not shown in cutaway initially (FIG. 6A), but is increasingly cutaway along a plane perpendicular to the instrument 301 as the tool enters (FIG. 6B) and moves through the vertebra (FIGS. 6C and 6D). In some aspects of the present technology, dynamically slicing the 3D image data 336 as the instrument 301 moves toward and/or through the 3D image data can provide the user with contextual information (e.g., an easy view of the walls of the vertebra) that is relevant to the position of the instrument 301. Moreover, for a user viewing the 3D image data 336 via a head-mounted display (e.g., the display 104 of FIG. 2) while operating the instrument 301, dynamically slicing the 3D image data 336 as shown can provide the user with an improved sense of embodiment and/or a tighter relationship between their body movements and the information being displayed-thereby providing more insight to the user with less effort.

In other embodiments, the position of the slicing plane can be determined/selected independently of the position of the tool. For example, FIGS. 7A-7D illustrate the 3D image data 336 including different slicing planes in accordance with embodiments of the present technology. Moreover, in the illustrated embodiment the slicing plane is obliquely angled relative to a longitudinal axis of the instrument 301 rather than orthogonal thereto. In some embodiments, such an oblique slicing plane can illustrate more perspective to the user, allowing for fewer changes to the perspective (e.g., coronal, axial, sagittal) of the 3D image data 336 during a procedure.

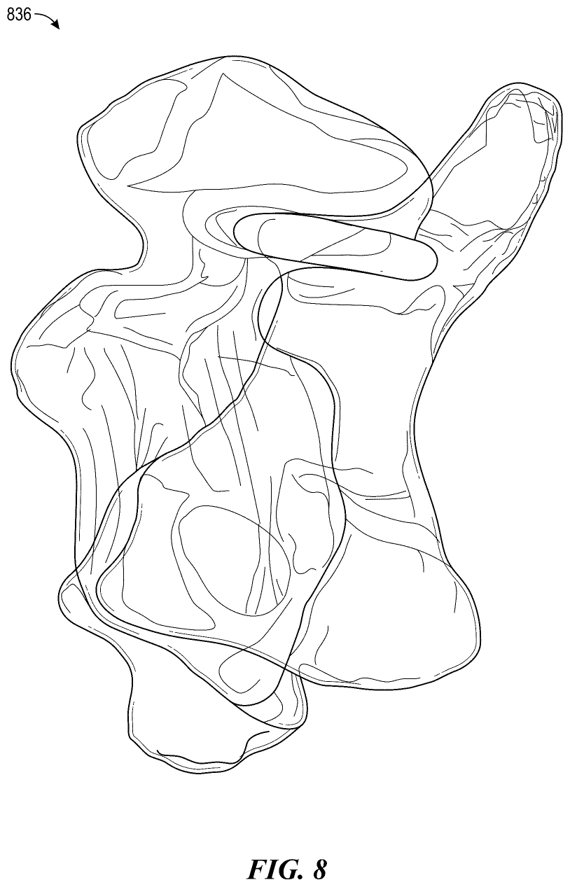

In other embodiments, the position and/or orientation of the slicing plane can be determined in other manners. For example, referring to FIGS. 1 and 2A, the system 100 can automatically select the position and/or orientation of the slicing plane based on a position of the display device 104 relative to the scene 108. For example, where the display device 104 is a head-mounted display device, the position and/or orientation of the slicing plane can be selected to correspond to a head position of a user (e.g., surgeon) wearing the head-mounted display device. In some embodiments, the display device 104 can include an eye tracker for tracking the user's eyes, and the position and/or orientation of the slicing plane can be determined based on the view direction of the user's eye In other embodiments, the slicing plane can be determined based on the position of one or more virtual cameras generated by the system 100. In some such embodiments, the slicing plane is aligned to be parallel with the virtual camera plane (e.g., parallel to a grid of pixels forming an image from the virtual camera) and at a predetermined distance relative to the 3D image data and/or relative to the virtual camera. When the virtual cameras move (e.g., via user input, tracking of the head of the user, etc.) the slicing plane can also move in 3D space to, for example, provide the user with a moving cutaway view around the 3D image data 336. In some embodiments, the user can select (e.g., via an icon, slider, or other feature on the user interface 330) a cutaway percentage of the 3D image data 336 that sets the predetermined distance of the slicing plane relative to the 3D image data 336. For example, at 0% cutaway, the slicing plane can be omitted such that the 3D image data 336 is not cutaway at all; at 30% cutaway, the slicing plane can be positioned 30% of the way along a length of the 3D image data 336 that is orthogonal to the virtual camera and from a surface of the 3D image data 336 (e.g., a surface nearest to the virtual camera); at 50% cutaway, the slicing plane can be positioned 50% of the way along a length of the 3D image data 336 that is orthogonal to the virtual camera and from a surface of the 3D image data 336 (e.g., a surface nearest to the virtual camera); at 100% cutaway, the entirety of the 3D image data can be shown as transparent or translucent (e.g., as shown in FIG. 8 below). Accordingly, the slicing plane can be positioned at a fixed depth relative to the 3D image data 336 to reveal a desired amount of the interior of the 3D image data 336, while still changing in orientation as the virtual camera changes in position and/or orientation.

As further shown in FIGS. 6A-7D, in some embodiments the portion of the 3D image data 336 that is cutaway can be shown as translucent (e.g., semi-transparent, “ghosted”). FIG. 8 is an enlarged illustration of 3D image data 836 of a vertebra shown as translucent in accordance with embodiments of the present technology. In some embodiments, the translucent view allows a user to view the contours and geometry of the vertebra without requiring additional cutaways or view changes. That is, such a translucent view can remove extraneous information while still allowing some information to peek through to the user around the boundaries of interest-for example, important information like curvature and boundaries are still visually available without the user having to seek an alternate viewing angle.

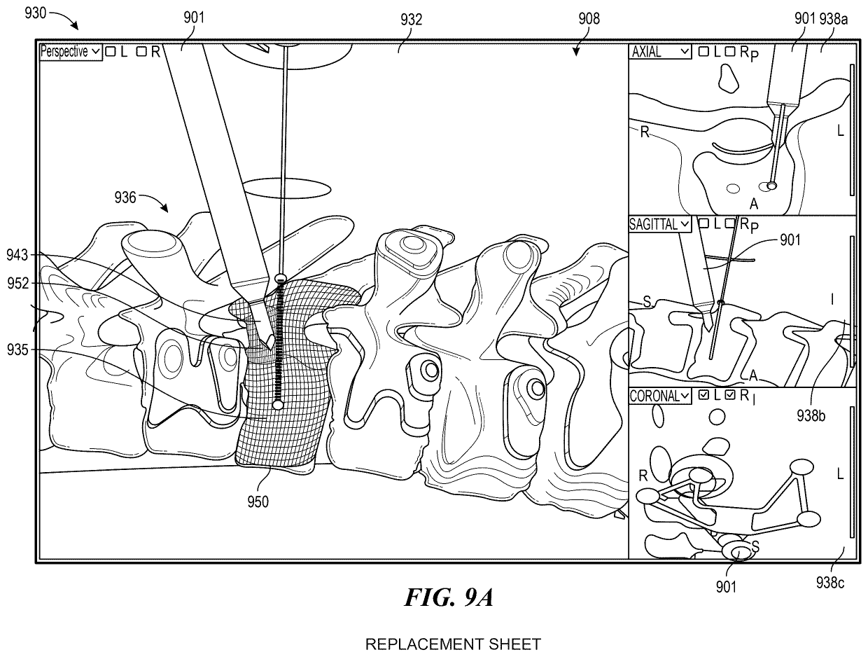

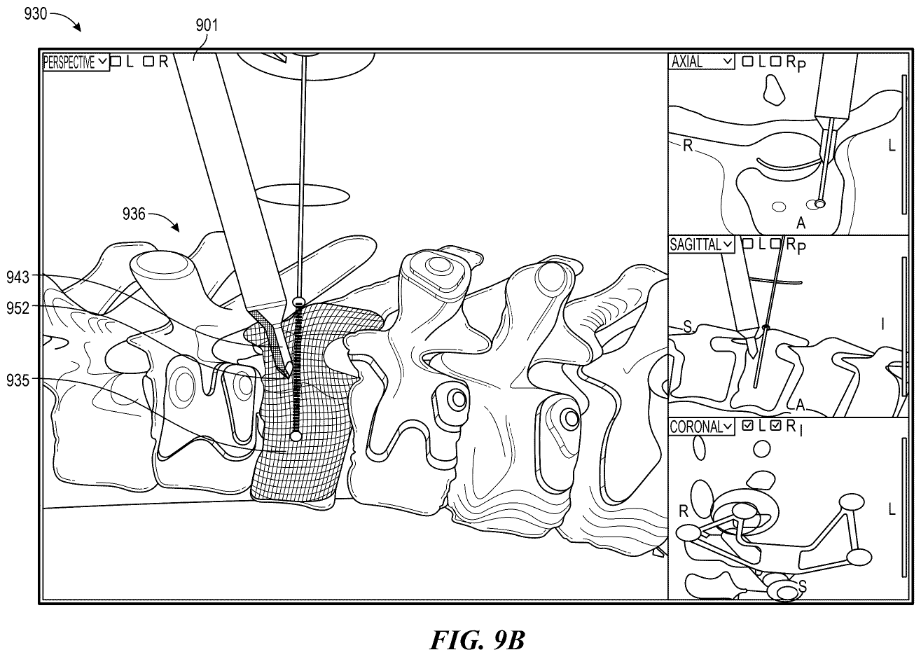

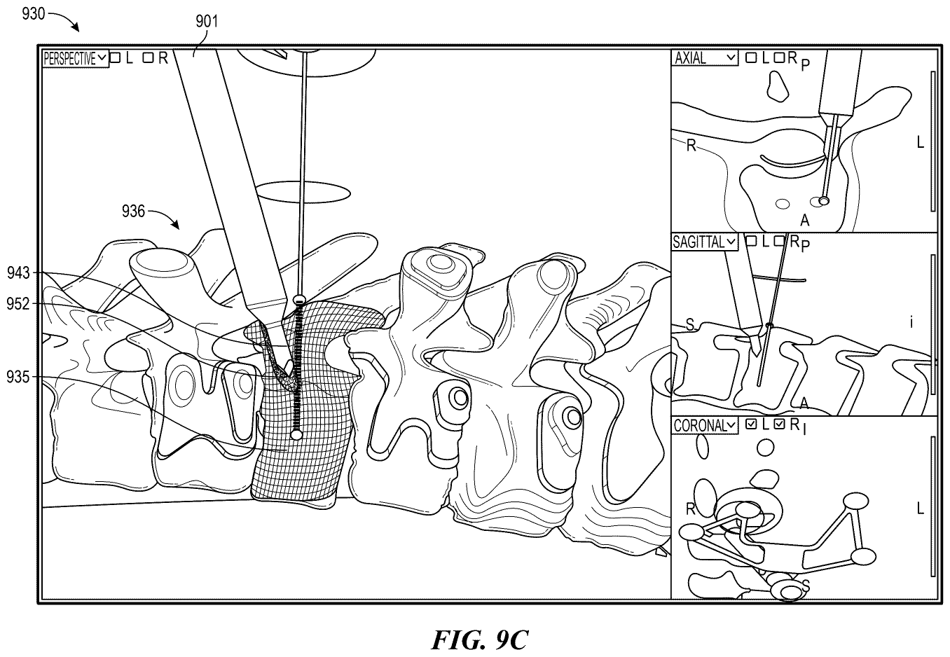

FIGS. 9A-9C illustrate a user interface (e.g., a display) 930 visible to a user of the system 100 via the display device 104 (e.g., a head-mounted display device) and/or the secondary display 226 in accordance with additional embodiments of the present technology. Referring first to FIG. 9A, the user interface 930 can include some features similar or identical to the user interface 330 described in detail above with reference to FIGS. 3-5D. For example, in the illustrated embodiment the user interface 930 includes a 3D view 932 and a plurality of 2D views 938 (identified individually as first through third 2D views 938a-c, respectively) of a physical scene 908 including an instrument 901. Previously-captured image data (e.g., CT scan data), is registered to the scene 908 and displayed on the 3D view 932 as 3D image data 936. The 2D views 938 can each display a 2D image corresponding to the 3D image data 936.

In the illustrated embodiment, the 3D image data 936 includes volumetric data of a patient's spine including a vertebra 950. Further, the instrument 901 is shown as inserted into the vertebra 950 during a procedure. In some embodiments, the instrument 901 can be a screw (e.g., a pedicle screw), a drill, and/or another tool used during a procedure to implant a screw or other implantable device in the vertebra 950. In some embodiments, it can be difficult for a user (e.g., a surgeon) operating the instrument 901 and viewing the user interface 930 to discern whether the instrument 901 is near a wall 935 of the vertebra 950. That is, for example, it can sometimes be difficult for the user to discern whether a tip 943 of the instrument 901 is likely to breach outside the wall 935 of the vertebra 950 based on the orientation and view perspective of the 3D image data 936—such as movement or positioning of the instrument 901 that may be occurring into or out of the projection plane of the image.

Accordingly, in the illustrated embodiment the user interface 930 includes a depth or breach indicator 952 configured to provide a visual indication on the 3D view 932 when the instrument 901 (e.g., the tip 943) is within a predefined distance from the wall 935 of the vertebra 950. More specifically, with additional reference to FIG. 1, the processing device 102 can (i) track the instrument 901 via information from the trackers 113, (ii) calculate a distance of the instrument 901 from the surface geometry of the wall 935 of the 3D image data 936, and (iii) compare the calculated distance to the predefined distance. In the illustrated embodiment, the breach indicator 952 includes highlighting (e.g., red highlighting) superimposed on the 3D image data 936, such as on the wall 935. The position of the breach indicator 952 can indicate where the instrument 901 is likely to breach the vertebra 950 if the user continues to move the instrument 901. In some embodiments, the highlighting can increase/decrease in brightness, color, and/or another characteristic as the instrument 901 moves closer to/farther from the wall 935.

In other embodiments, the breach indicator 952 can include other types of visual cues. In FIG. 9B, for example, the breach indicator 952 comprises highlighting on the instrument 901, such as on the tip 943 of the instrument 901. In some embodiments, the highlighting on the instrument 901 can indicate a directionality of the instrument 901 relative to the closest portion of the wall 935 of the 3D image data 936 (e.g., to a potential exit point or area of potential breach). Similarly, in FIG. 9C, the breach indicator 952 includes highlighting on both the tip 943 of the instrument 901 and the wall 935 of the 3D image data 936. Accordingly, in some aspects of the present technology, the system 100 can utilize the 3D view 932 to provide real-time depth feedback to the user via different visual cues of the breach indicator 952. In some aspects of the present technology, overlaying depth information on the 3D image data 936 can reduce the need for the user to refer to multiple projections from different vantage points, such as the 2D views 938, to determine depth and positioning.

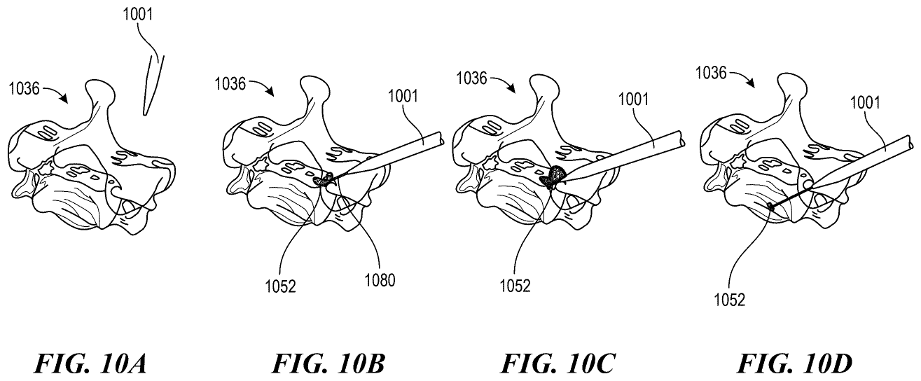

FIGS. 10A-10D illustrate 3D image data 1036 and an instrument 1001 approaching, entering, and moving through the 3D image data 1036 (and registered physical vertebra) in accordance with embodiments of the present technology. Referring first to FIG. 10A, the instrument 1001 has yet to enter the vertebra and no breach indicator or instrument trajectory is displayed. FIG. 10B illustrates the instrument 1001 contacting an entry point on the vertebra. In the illustrated embodiment, after contacting the vertebra with the instrument 1001, the system 100 can display a projected trajectory 1080 and a breach indicator 1052 comprising highlighting on a portion of the 3D image data 1036 where a breach may occur if the instrument 1001 continues along the projected trajectory 1080. In some aspects of the present technology, the projected trajectory 1080 and the breach indicator 1052 allow the user to visualize a path—and anatomy along the path—to enable the user to select a desired angle of entry. For example, FIG. 10D illustrates a change of angle in the instrument 1001 such that the breach indicator 1052 is positioned on a farther wall of the 3D image data 1036. In some embodiments, the breach indicator 1052 can have a reduced size based on a distance to breach (e.g., having a smaller size in FIG. 10D than FIG. 10B). FIG. 10C illustrates the instrument 1001 breaching the vertebra, such as if the user had not corrected the angle of entry from that shown in FIG. 10B. In some embodiments, after breach (and/or immediately before breach), the breach indicator 1052 can change color, intensity, size, and/or other characteristics to indicate to the user that breach has occurred or is imminent. Accordingly, in some aspects of the present technology the breach indicator 1052 can provide a prediction of a breach location and likelihood, and/or an indication that breach as occurred (e.g., a breach detection).

Referring to FIGS. 1-10D together, in some embodiments the geometry of the physical scene can change relative to the previously-captured image data displayed on the user interface. For example, during a surgical procedure, the surgeon may remove a portion of bone or tissue. Specifically, during spinal surgical procedures, surgeons often burr away a portion of the vertebra before placing hardware or other implants. After removing the bone or tissue, the physical geometry of the scene will not exactly match previously-captured image data, such as CT data of the region of bone or tissue. That is, for example, the images of the patient's vertebra presented on the 3D views 332, 932 and/or the 2D views 338, 938—which are generated before the surgical procedure—may not correspond to the physical geometry of the vertebra during the procedure. Accordingly, depth information calculated from the previously-captured image data may not accurately represent the geometry of the scene.

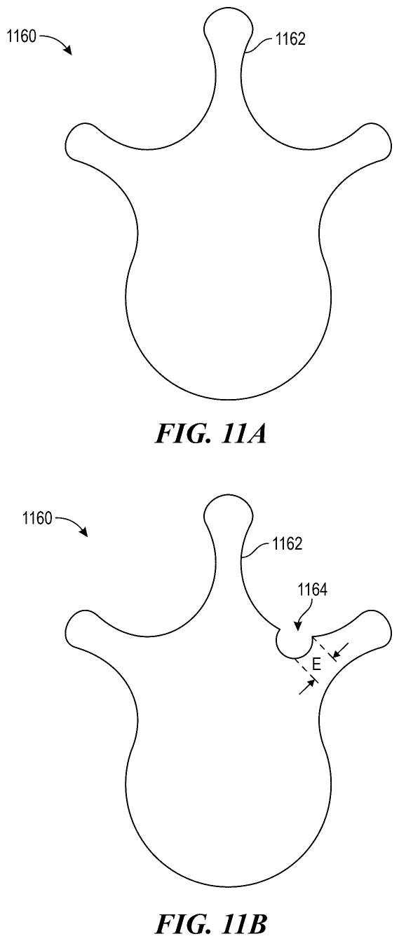

More specifically, FIGS. 11A and 11B are schematic representations of a vertebra 1160 of a patient having a surface 1162 in accordance with embodiments of the present technology. As shown in FIG. 7B, a surgeon may remove a portion 1164 of the vertebra 1160 such that the surface 1162 of the vertebra 1160 changes. If depth measurements are calculated based on CT data or other previously-captured image data of the vertebra 1160 before the portion 1164 is removed, the depth measurements may not correspond to an actual depth of the vertebra 1160. That is, for example, the depth measurements can have an error E corresponding to a dimension of the portion 1164 removed from the vertebra 1160.

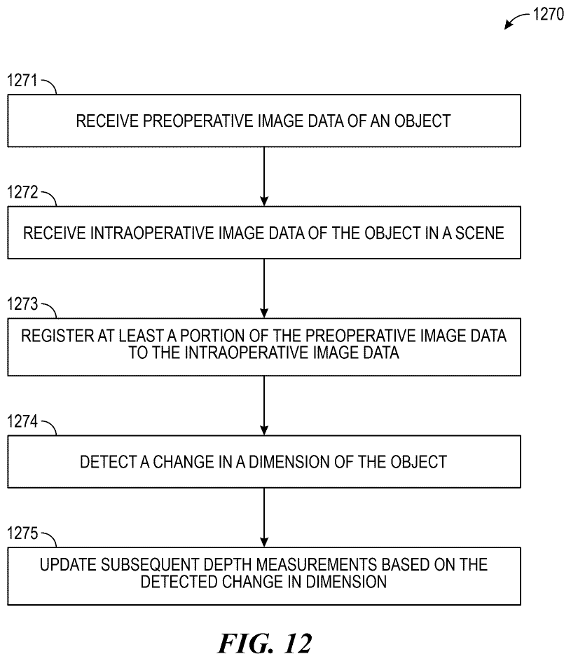

FIG. 12 is a flow diagram of a process or method 1270 for updating depth information of a physical scene (e.g., including the anatomy of patient) after the scene changes in accordance with embodiments of the present technology. Although some features of the method 1270 are described in the context of the system 100 shown in FIGS. 1-2B for the sake of illustration, one skilled in the art will readily understand that the method 1270 can be carried out using other suitable systems and/or devices described herein. Similarly, while reference is made herein to preoperative image data, intraoperative image data, and a surgical scene, the method 1270 can be used with other types of information about other scenes.

At block 1271, the method 1270 includes receiving preoperative image data of an object. As described in detail above, the preoperative image data can be, for example, medical scan data representing a three-dimensional volume of a patient, such as computerized tomography (CT) scan data, magnetic resonance imaging (MRI) scan data, ultrasound images, fluoroscope images, and the like. In some embodiments, the preoperative image data can comprise a point cloud or 3D mesh. The object can be patient's vertebra, spine, knee, skull, and/or the like.

At block 1272, the method 1270 includes receiving intraoperative image data of the object in the scene 108 from, for example, the camera array 110. The intraoperative image data can include real-time or near-real-time images of a patient in the scene 108 captured by the cameras 112 and/or the depth cameras 118. In some embodiments, the intraoperative image data includes (i) light field images from the cameras 112 and/or (ii) images from the depth cameras 118 that include encoded depth information about the scene 108. In some embodiments, the preoperative image data corresponds to at least some features in the intraoperative image data. For example, the scene 108 can include a patient undergoing spinal surgery with their spine at least partially exposed. The preoperative image data can include CT scan data of the patient's spine taken before surgery and that comprises a complete 3D data set of at least a portion of the spine. Accordingly, various vertebrae or other features in the preoperative image data can correspond to portions of the patient's spine represented in the image data from the cameras 112, 118. In other embodiments, the scene 108 can include a patient undergoing another type of surgery, such as knee surgery, skull-based surgery, and so on, and the preoperative image data can include CT or other scan data of ligaments, bones, flesh, and/or other anatomy relevant to the particular surgical procedure.

At block 1273, the method 1270 includes registering the preoperative image data to the intraoperative image data to, for example, establish a transform/mapping/transformation between the intraoperative image data and the preoperative image data so that these data sets can be represented in the same coordinate system. The registration can include a global registration and/or one or more refined (e.g., local) registrations. In some embodiments, the method 1270 can include registering the preoperative image data to the preoperative image data using any of the methods disclosed in U.S. patent application Ser. No. 17/140,885, titled “METHODS AND SYSTEMS FOR REGISTERING PREOPERATIVE IMAGE DATA TO INTRAOPERATIVE IMAGE DATA OF A SCENE, SUCH AS A SURGICAL SCENE,” and filed Jan. 4, 2021, which is incorporated herein by reference in its entirety.

At block 1274, the method 1270 includes detecting a change in a dimension in the object. In some embodiments, the system 100 can detect the change in dimension as a change in depth captured by the depth cameras 118 and/or the cameras 112 (e.g., based on light field image data captured by the cameras 112). For example, referring to FIGS. 11A and 11B, in some embodiments the system 100 can automatically detect that the geometry of the surface 1162 has changed due to the removal of the portion 1164.

At block 1275, the method 1270 includes updating subsequent depth measurements of the object based on the detected change in dimension. In some embodiments, the system 100 can update the preoperative image data to reflect the change in dimension in the object. For example, where the preoperative image data comprises a 3D mesh, the system 100 can update the mesh to reflect the intraoperative change in dimension. In such embodiments, subsequent depth measurements based on the 3D mesh will reflect the change in dimension. Alternatively, the system 100 can simply “zero-out” any depth measurements taken where the dimension of the object changed such that, for example, the depth readout 448 shown in FIGS. 4A-5B indicates a correct zero depth when the instrument 301 is positioned near the change in dimension (e.g., at a burred region). Accordingly, in some aspects of the present technology the method 1270 can include updating depth measurements and/or the preoperative image data to more accurately represent intraoperative changes in anatomy.

Additionally or alternatively, the method 1270 can include updating a surgical plan based on the detected change in dimension of the object. For example, a predetermined entry point for a surgical implant (e.g., a pedicle screw) can be changed to avoid the area of changed dimension. Similarly, a dimension of the implant (e.g., a length, width) and/or angle of entry can be updated based on the detected change in dimension.

FIGS. 13A and 13B are an axial cutaway view and an oblique cutaway view, respectively, of the 3D image data 336 on the user interface 330 of FIG. 3 illustrating the overlay of preoperative plan information in accordance with embodiments of the present technology. Referring to FIGS. 13A and 13B together, the preoperative plan information can include a trajectory 1390 for an implant 1391 (e.g., a pedicle screw) through the vertebra. In the illustrated embodiment, the preoperative plan information further includes one or more angle-of-entry indicators 1392 (identified individually as a first angle-of-entry indicator 1392a and a second angle-of-entry indicator 1392b). As best seen in FIG. 13B, the angle-of-entry indicators 1392 can include one or more circles or rings that are concentric with the trajectory 1390. In operation, the user can align the instrument 301 within the angle-of-entry indicators 1392 to orient the instrument 301 and the implant 1391 at the correct (pre-selected) angle along the trajectory 1390. The trajectory 1390 and angle-of-entry indicators can be fixed (e.g., locked, stationary) relative to the 3D image data 336 and, in some embodiments, can be adjusted by the user during a procedure to place the implant 1391. In some embodiments, the trajectory 1390 and the angle-of-entry indicators 1392 can also be displayed in one or more of the 2D views 338.

In the illustrated embodiment, the trajectory 1390 has an endpoint 1394 within the vertebra. The user interface 330 can further display a depth readout 1396 indicating a depth (e.g., a distance) of the implant 1391 relative to a determined target depth of the endpoint 1394 (“40.0 mm”). The depth can be updated in real time or near real time. In FIG. 13A, the implant 1391 has been advanced to a first depth (“22.5 mm”) within the vertebra using the instrument 1301. In FIG. 13B, the implant 1391 has been advanced to a second depth (“42.5 mm”) within the vertebra. As shown in FIG. 13B, the depth readout 1396 can provide an alert (e.g., larger font, changed color, and so on) when the depth of the implant 1391 matches and/or exceeds the predetermined target depth of the implant 1391 (e.g., “40.0 mm”).

During insertion of the implant 1391, the cutaway views can allow the user to determine how much clearance there is between the implant 1391 and the walls of the vertebra to help avoid breach. Moreover, in some embodiments the depth readout 1396 can be anchored to the wall of the 3D image data 336 so as not to clutter or interfere with the user's view of the trajectory 1390.

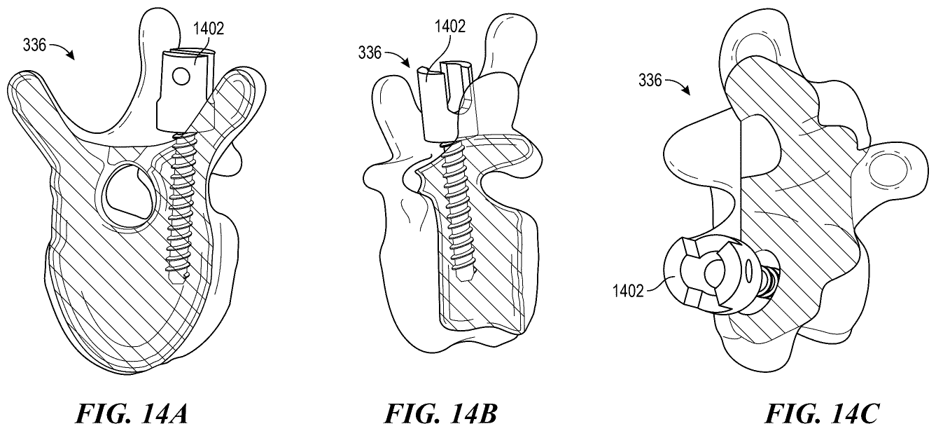

FIGS. 14A-14C are an axial, sagittal, and coronal cutaway view, respectively, of the 3D image data 336 of FIG. 3 illustrating the overlay of a 3D representation of an implant 1402 in accordance with embodiments of the present technology. Referring to FIGS. 14A-14C together, in the illustrated embodiment the implant is a screw, such as a pedicle screw. In some embodiments, the 3D representation of the implant 1402 can be superimposed on the 3D image data 336 preoperatively (e.g., based on a preoperative plan) or intraoperatively. When positioned intraoperatively, the position of the 3D representation of the implant 1402 can be based on a user-selected entry point and/or based on a tracked-position of the instrument 301 (FIG. 3). For example, with additional reference to FIG. 1, the trackers 113 can track the instrument 301, and the system 100 can position the 3D representation of the implant 1402 based on a known size (e.g., width and length) of the implant and the standard positioning of the instrument 301 relative to the implant. In some aspects of the present technology, intraoperatively overlaying the 3D representation of the implant 1402 (e.g., after physical placement of the implant) can allow a user to explore the vertebra to look for breaches in the placement of the implant. In additional aspects of the present technology, preoperatively overlaying the 3D representation of the implant 1402 can help guide the user to a target placement before the procedure has begun. Further, the cutaway views can allow the user to observe the placement of the implant with fewer needs for perspective changes.

### III. Additional Examples

The following examples are illustrative of several embodiments of the present technology:
1. A method of displaying three-dimensional (3D) image data on a user interface, the method comprising:
registering the 3D image data to a physical scene;
tracking an instrument through the physical scene;
displaying the 3D image data and a representation of the instrument on the user interface; and
displaying a cross-section of the 3D image data.
2. The method of example 1 wherein the method further comprises determining a position of the cross-section relative to the 3D image data based on a tracked position of the instrument.
3. The method of example 2 wherein displaying the cross-section including displaying the cross-section oriented perpendicular to a longitudinal axis of the instrument.
4. The method of any one of examples 1-3 wherein the method further comprises: capturing image data of the physical scene with a camera array;
synthesizing a virtual image corresponding to a perspective of a virtual camera based on the image data from the camera array; and
determining a position of the cross-section relative to the 3D image data based on the perspective of the virtual camera.
5. The method of example 4 wherein determining the position of the cross-section includes determining the position to be at a predetermined distance from the perspective of the virtual camera.
6. The method of example 4 wherein determining the position of the cross-section includes determining the position to be at a set depth relative to the 3D image data.
7. The method of any one of examples 4-6 wherein displaying the cross-section includes displaying the cross-section oriented parallel to the perspective of the virtual camera.
8. The method of any one of examples 1-7 wherein displaying the cross-section of the 3D image data includes displaying a physical geometry of an inner surface of an object represented in the 3D image data.
9. The method of example 8 wherein the object is a vertebra.
10. The method of any one of examples 1-9 wherein the 3D image data includes computed tomography (CT) data, and wherein the 3D image data is of a portion of a patient's spine.
11. A method of displaying three-dimensional (3D) image data on a user interface, the method comprising:
registering the 3D image data to a physical scene, wherein the 3D image data defines a surface;
tracking an instrument through the physical scene;
displaying the 3D image data and a representation of the instrument on the user interface;
calculating a distance between the instrument and the surface; and
displaying the distance on the user interface.
12 The method of example 11 wherein calculating the distance includes calculating the distance in real-time, and wherein displaying the distance includes displaying the real-time distance.
13. The method of example 11 or example 12 wherein the distance is a distance between a tip of the instrument and the surface of the of the 3D image data along a longitudinal axis of the instrument.
14. The method of any one of examples 11-13 wherein the surface is an interior surface of the 3D image data.
15 The method of any one of examples 11-13 wherein the surface is an exterior surface of the 3D image data.
16. The method of any one of examples 11-15 wherein the method further comprises receiving a known distance between the instrument and the surface before calculating the distance between the instrument and the surface.
17. The method of example 16 where the known distance is zero.
18. The method of any one of examples 11-17 wherein the distance is a depth of a tip of the instrument below the surface of the 3D image data.
19. The method of any one of examples 11-18 wherein the method further comprises displaying an indication of a likelihood and/or a predicted location that the instrument could breach the surface of the 3D image data.
20. The method of example 19 wherein the method further comprises determining the likelihood and/or the predicted location based on the distance.
21. The method of example 19 or example 20 wherein displaying the indication includes highlighting a portion of the 3D image data on the user interface.
22. The method of any one of examples 11-21 wherein the instrument is a surgical tool.
23. The method of any one of examples 11-21 wherein the instrument is a surgical implant.
24. The method of any one of examples 11-21 wherein the instrument is a surgical tool coupled to a surgical implant.
25. The method of any one of examples 11-24 wherein the method further comprises displaying an indication that the instrument has breached the surface of the 3D image data.
26. The method of example 25 wherein the method further comprises determining that the instrument has breached the surface of the 3D image data based on the distance.
27. A method of displaying three-dimensional (3D) image data on a user interface, the method comprising:
registering the 3D image data to a physical scene, wherein the 3D image data defines a surface;
tracking an instrument through the physical scene;
displaying the 3D image data and a representation of the instrument on the user interface;
calculating a distance between the instrument and the surface; and
displaying an indication on the user interface when the distance is less than a predefined threshold.
28. The method of example 27 wherein displaying the indication includes highlighting a portion of the 3D image data on the user interface.
29 The method of example 27 or example 28 wherein displaying the indication includes highlighting a portion of the representation of the instrument on the user interface.
30. The method of any one of examples 27-29 wherein the indication indicates a likelihood and/or a predicted location that the instrument could breach the surface of the 3D image data.
31. The method of any one of examples 27-29 wherein the indication indicates that the instrument has breached the surface of the 3D image data.
32. The method of any one of examples 27-31 wherein the instrument is a surgical tool.
33. The method of any one of examples 27-31 wherein the instrument is a surgical implant.
34. The method of any one of examples 27-31 wherein the instrument is a surgical tool coupled to a surgical implant.
35. A method of updating preoperative three-dimensional (3D) image data of an object, the method comprising:
registering the preoperative 3D image data to the object;
capturing intraoperative depth data of the object;
detecting a change in dimension of the object in the depth data; and
updating the preoperative 3D image data based on the detected change in dimension.
36. The method of example 35 wherein detecting the change in dimension of the object includes detecting that a portion of the object has been removed.
37. The method of example 35 or example 36 wherein the preoperative 3D image data includes a 3D mesh, and wherein updating the preoperative 3D image data includes updating the 3D mesh to reflect the change in dimension.
38. An imaging system, comprising:
a camera array including a plurality of cameras configured to capture intraoperative image data; and
a non-transitory computer-readable storage medium storing instructions that, when executed by one or more processors, cause the imaging system to perform operations comprising any one of examples 1-37.
38. A non-transitory computer-readable storage medium storing instructions that, when executed by one or more processors, cause an imaging system to perform operations comprising any one of examples 1-37.

### IV. Conclusion

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1-25. (canceled)

26. A method of updating three-dimensional (3D) image data of an object, the method comprising:
registering the 3D image data to the object;
capturing intraoperative depth data of the object;
detecting a change in dimension of the object in the depth data; and
updating the 3D image data based on the detected change in dimension.

27. The method of claim 26 wherein detecting the change in dimension of the object comprises detecting that a portion of the object has been removed.

28. The method of claim 26 wherein the 3D image data includes a 3D mesh, and wherein updating the 3D image data comprises updating the 3D mesh to reflect the change in dimension.

29. The method of claim 26 wherein the method further comprises:
displaying the 3D image data on a user interface after registering the 3D image data; and
displaying the updated 3D image data on the user interface after updating the 3D image data.

30. The method of claim 26 wherein registering the 3D image data to the object is based at least in part on the intraoperative depth data.

31. The method of claim 26 wherein the object is a vertebra.

32. A method of updating three-dimensional (3D) image data of a patient a surgical procedure on the patient within a surgical scene, the method comprising:
capturing, with a camera array positioned to view the surgical scene and external to the patient, intraoperative image data of the surgical scene;
registering the 3D image data to the surgical scene based on the intraoperative image data;
detecting a change in dimension in the intraoperative image data; and
updating the 3D image data based on the detected change in dimension.

33. The method of claim 32 wherein the method further comprises displaying the updated 3D image data on a user interface.

34. The method of claim 32 wherein the method further comprises:
tracking a surgical instrument through the surgical scene based on the intraoperative image data, wherein the surgical instrument is a surgical tool, a surgical implant, or a surgical tool coupled to a surgical implant; and
displaying the 3D image data and a representation of the surgical instrument on the user interface in real-time or near real-time, wherein the representation of the surgical instrument indicates a tracked position of the surgical instrument relative to the patient.

35. The method of claim 32 wherein the method further comprises:
displaying a cross-section of the updated 3D image data on a user interface in real-time or near real-time, wherein displaying the cross-section of the updated 3D image includes determining a position of the cross-section relative to the updated 3D image data based on the tracked position of the surgical instrument; and
continuously updating the position of the cross-section based on the tracked position of the surgical instrument.

36. The method of claim 32 wherein the 3D image data includes computed tomography (CT) data, and wherein the 3D image data is of a portion of a spine of the patient.

37. £ (New) The method of claim 32 wherein the 3D image data includes computed tomography (CT) data, and wherein the 3D image data is of a portion of a spine of the patient.

38. The method of claim 37 wherein detecting the change in dimension in the 3D image data object comprises detecting that a portion of the spine has been removed.

39. The method of claim 32 wherein detecting the change in dimension in the 3D image data object comprises detecting that a portion of the patient has been removed.

40. The method of claim 32 wherein the 3D image data includes a 3D mesh, and wherein updating the 3D image data comprises updating the 3D mesh to reflect the change in dimension.

41. The method of claim 32 wherein the method further comprises updating a surgical plan based on the updated 3D image data.

42. The method of claim 41 wherein updating the surgical plan comprsises changing a predetermined entry point for a surgical implant.

43. The method of claim 41 wherein updating the surgical plan comprsises changing a dimension of a surgical implant.
